(19) United States
(12) Patent Application Publication
(10) Pub. No.: US 2011/0099670 A1
(43) Pub. Date: Apr. 28, 2011

(54) NUCLEOTIDE SEQUENCES CODING FOR CIS-ACONITIC DECARBOXYLASE AND USE THEREOF

(76) Inventors: Andries Jurriaan Koops, Opheusden (NL); Leendert Hendrik De Graaff, Arnhem (NL); Ingrid Maria Van Der Meer, Wageningen (NL); Wilhelmus Antonius Maria Van Den Berg, Renkum (NL)

(21) Appl. No.: 12/867,030

(22) PCT Filed: Feb. 13, 2009
(86) PCT No.: PCT/NL09/50065

§ 371 Date: Dec. 13, 2010

(30) Foreign Application Priority Data
Feb. 14, 2008 (EP) 08151452.3

(51) Int. Cl. C12P 7/44; C12N 9/88; C12N 15/00; C12N 5/10; C12N 1/15; C12N 1/19; A01H 5/00; C07C 57/13

(52) U.S. Cl. 800/298; 536/23.2; 435/320.1; 435/419; 435/254.11; 435/254.3; 435/254.5; 435/254.22; 435/142; 562/590

(57) ABSTRACT

The present invention relates to nucleotide sequences encoding polypeptides with cis-aconitic decarboxylase activity, the cells transformed with such nucleotide sequences, preferably fungal or plant cells, and to methods wherein such transformed cells are use for the production of itaconic acid.

### FIELD OF THE INVENTION

The present invention relates to nucleotide sequences coding for cis-aconitic decarboxylases and to the use of these sequence for the production of itaconic acid in genetically modified microorganisms and transgenic plants that express the cis-aconitic decarboxylases encoding sequences.

### BACKGROUND OF THE INVENTION

Itaconic acid is a C5 dicarboxylic acid, also known as methyl succinic acid. Itaconic acid has the potential to be a key building block for deriving both commodity and specialty chemicals. The basic chemistry of itaconic acid is similar to that of the petrochemicals derived from maleic acid/anhydride. Being able to do various kinds of addition-, esterification- and polymerization-reactions, it is an important material for the chemical synthetic industry as well as for the production of chemical intermediates.

Currently, itaconic acid is used as a co-monomer in acrylic fibres and styrene materials to aid the dyeing and painting properties. Acrylic fibers, which have included itaconic acid as the third monomer, are much easier to dye. Itaconic acid is also used to improve the optical properties of plastics. Polymers which contain itaconic acid have special transparency and lustre qualities.

The problem of current itaconic acid manufacturing is the high production cost, thus limiting the use of this promising biological molecule as a building block for high value chemical intermediates and polymers. Should the price of itaconic acid be reduced then it is reasonable to expect more applications in the area of bio-based chemical building blocks.

Itaconic acid can be produced chemically by the pyrolysis of citric acid, resulting in waterloss and conversion of citric acid in aconitate. Subsequent decarboxylation of aconitate gives two isomers itaconic acid and citraconic acid. This chemical synthesis route of itaconic acid has proven uneconomical for a number of reasons, including the relatively high substrate costs, the low yields and the co-production of various other acids such as succinic acid and tartaric acid (Brian Currell, R. C.; Van Dam Mieras; Biotol Partners Staff; 1997; Biotechnological Innovations in Chemical Synthesis. Elsevier).

A currently more promising production route is via fungal fermentation. Itaconic acid is commercially produced by Aspergillus terreus. The global production volume remains relatively low (estimated to be ca. 5000-10000 tonnes per annum) and the price relatively high (ca. [symbol] 2500-4000 per tonne). Though fungal fermentation is economically a more viable route compared to chemical production, the cost price of also the fungal production is still a major hurdle for the development of itaconic acid as a building block for commodity chemicals.

It is thus an object of the present invention to provide for means and methods that allow for a more cost effective production of itaconic acid.

### DESCRIPTION OF THE INVENTION

### Definitions

The term “nucleic acid sequence” (or nucleic acid molecule) refers to a DNA or

RNA molecule in single or double stranded form, particularly a DNA having promoter activity according to the invention or a DNA encoding a protein or protein fragment. An “isolated nucleic acid” refers to a nucleic acid which is no longer in the natural environment from which it was isolated, e.g. the nucleic acid sequence in a fungal host cell or in the plant nuclear or plastid genome.

The term peptide herein refers to any molecule comprising a chain of amino acids that are linked in peptide bonds. The term peptide thus includes oligopeptides, polypeptides and proteins, including multimeric proteins, without reference to a specific mode of action, size, 3-dimensional structure or origin. The terms “protein” or “polypeptide” are used interchangeably. A “fragment” or “portion” of a protein may thus still be referred to as a “protein”. An “isolated protein” is used to refer to a protein which is no longer in its natural environment, for example in vitro or in a recombinant (fungal or plant) host cell. The term peptide also includes post-expression modifications of peptides, e.g. glycosylations, acetylations, phosphorylations, and the like.

The term “gene” means a DNA sequence comprising a region (transcribed region), which is transcribed into an RNA molecule (e.g. an mRNA) in a cell, operably linked to suitable transcription regulatory regions (e.g. a promoter).

A gene may thus comprise several operably linked sequences, such as a promoter, a 5′ non-translated leader sequence (also referred to as 5′UTR, which corresponds to the transcribed mRNA sequence upstream of the translation start codon) comprising e.g. sequences involved in translation initiation, a (protein) coding region (cDNA or genomic DNA) and a 3′non-translated sequence (also referred to as 3′ untranslated region, or 3′UTR) comprising e.g. transcription termination sites and polyadenylation site (such as e.g. AAUAAA or variants thereof).

A “chimeric gene” (or recombinant gene) refers to any gene, which is not normally found in nature in a species, in particular a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more sense sequences (e.g. coding sequences) or to an antisense (reverse complement of the sense strand) or inverted repeat sequence (sense and antisense, whereby the RNA transcript forms double stranded RNA upon transcription).

A “3′ UTR” or “3′ non-translated sequence” (also often referred to as 3′ untranslated region, or 3′ end) refers to the nucleic acid sequence found downstream of the coding sequence of a gene, which comprises, for example, a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

“Expression of a gene” refers to the process wherein a DNA region, which is operably linked to appropriate regulatory regions, particularly a promoter, is transcribed into a RNA, which is biologically active, i.e. which is capable of being translated into a biologically active protein or peptide (or active peptide fragment) or which is active itself (e.g. in posttranscriptional gene silencing or RNAi, or silencing through miRNAs). The coding sequence is preferably in sense-orientation and encodes a desired, biologically active protein or peptide, or an active peptide fragment.

“Ectopic expression” refers to expression in a tissue in which the gene is normally not expressed.

A “transcription regulatory sequence” is herein defined as a nucleic acid sequence that is capable of regulating the rate of transcription of a nucleic acid sequence operably linked to the transcription regulatory sequence. A transcription regulatory sequence as herein defined will thus comprise all of the sequence elements necessary for initiation of transcription (promoter elements), for maintaining and for regulating transcription, including e.g. attenuators or enhancers, but also silencers. Although mostly the upstream (5′) transcription regulatory sequences of a coding sequence are referred to, regulatory sequences found downstream (3′) of a coding sequence are also encompassed by this definition.

As used herein, the term “promoter” refers to a nucleic acid fragment that functions to control the transcription of one or more genes, located upstream (5′) with respect to the direction of transcription of the transcription initiation site of the gene (the transcription start is referred to as position +1 of the sequence and any upstream nucleotides relative thereto are referred to using negative numbers), and is structurally identified by the presence of a binding site for DNA-dependent RNA polymerase, transcription initiation sites and any other DNA domains (cis acting sequences), including, but not limited to transcription factor binding sites, repressor and activator protein binding sites, and any other sequences of nucleotides known to one of skill in the art to act directly or indirectly to regulate the amount of transcription from the promoter. Examples of eukaryotic cis acting sequences upstream of the transcription start (+1) include the TATA box (commonly at approximately position −20 to −30 of the transcription start), the CAAT box (commonly at approximately position −75 relative to the transcription start), 5′ enhancer or silencer elements, etc. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells.

As used herein, the term “operably linked” refers to a linkage of polynucleotide elements in a functional relationship. A nucleic acid is “operably linked” when it is placed into a functional relationship with another nucleic acid sequence.

For instance, a promoter, or a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which are joined to form a functional protein, which displays the functionality of the joined domains (for example a DNA binding domain or a repression of function domain leading to a dominant negative function). A chimeric protein may also be a fusion protein of two or more proteins occurring in nature. The term “domain” as used herein means any part(s) or domain(s) of the protein with a specific structure or function that can be transferred to another protein for providing a new hybrid protein with at least the functional characteristic of the domain.

A “nucleic acid construct” is herein understood to mean a man-made nucleic acid molecule resulting from the use of recombinant DNA technology. A nucleic acid construct is a nucleic acid molecule, either single- or double-stranded, which has been modified to contain segments of nucleic acids, which are combined and juxtaposed in a manner, which would not otherwise exist in nature. A nucleic acid construct usually is a “vector”, i.e. a nucleic acid molecule which is used to deliver exogenously created DNA into a host cell. Vectors usually comprise further genetic elements to facilitate their use in molecular cloning, such as e.g. selectable markers, multiple cloning sites and the like.

One type of nucleic acid construct is an “expression cassette” or “expression vector”. These terms refers to nucleotide sequences that are capable of effecting expression of a gene in host cells or host organisms compatible with such sequences. Expression cassettes or expression vectors typically include at least suitable transcription regulatory sequences and optionally, 3′ transcription termination signals. Additional factors necessary or helpful in effecting expression may also be present, such as expression enhancer elements. DNA encoding the polypeptides of the present invention will typically be incorporated into the expression vector. The expression vector will be introduced into a suitable host cell and be able to effect expression of the coding sequence in an in vitro cell culture of the host cell. The expression vector preferably is suitable for replication in a fungal, plant and/or in a prokaryotic host.

A “host cell” or a “recombinant host cell” or “transformed cell” are terms referring to a new individual cell (or organism), arising as a result of the introduction into said cell of at least one nucleic acid construct, especially comprising a chimeric gene encoding a desired protein. The host cell may be a plant cell, a bacterial cell, a fungal cell (including a yeast cell), etc. The host cell may contain the nucleic acid construct as an extra-chromosomally (episomal) replicating molecule, or more preferably, comprises the chimeric gene integrated in the nuclear or plastid genome of the host cell.

The term “selectable marker” is a term familiar to one of ordinary skill in the art and is used herein to describe any genetic entity which, when expressed, can be used to select for a cell or cells containing the selectable marker. Selectable markers may be dominant or recessive or bidirectional. The selectable marker may be a gene coding for a product which confers antibiotic or herbicide resistance to a cell expressing the gene or a non-antibiotic marker gene, such as a gene relieving other types of growth inhibition, i.e. a marker gene which allow cells containing the gene to grow under otherwise growth-inhibitory conditions. Examples of such genes include a gene which confers prototrophy to an auxotrophic strain. The term “reporter” is mainly used to refer to visible markers, such as green fluorescent protein (GFP), eGFP, luciferase, GUS and the like, as well as nptII markers and the like.

The term “ortholog” of a gene or protein refers herein to the homologous gene or protein found in another species, which has the same function as the gene or protein, but (usually) diverged in sequence from the time point on when the species harbouring the genes diverged (i.e. the genes evolved from a common ancestor by speciation). Orthologs of a gene from one species may thus be identified in other species based on both sequence comparisons (e.g. based on percentages sequence identity over the entire sequence or over specific domains) and functional analysis.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's).

“Self-cloning” is defined herein as in European Directive 98/81/EC Annex II: Self-cloning consists in the removal of nucleic acid sequences from a cell of an organism which may or may not be followed by reinsertion of all or part of that nucleic acid (or a synthetic equivalent) with or without prior enzymic or mechanical steps, into cells of the same species or into cells of phylogenetically closely related species which can exchange genetic material by natural physiological processes where the resulting micro-organism is unlikely to cause disease to humans, animals or plants. Self-cloning may include the use of recombinant vectors with an extended history of safe use in the particular micro-organisms.

When used to indicate the relatedness of two nucleic acid sequences the term “homologous” means that one single-stranded nucleic acid sequence may hybridise to a complementary single-stranded nucleic acid sequence. The degree of hybridisation may depend on a number of factors including the amount of identity between the sequences and the hybridisation conditions such as temperature and salt concentration as discussed later.

“Stringent hybridisation conditions” can be used to identify nucleotide sequences, which are substantially identical to a given nucleotide sequence. The stringency of the hybridization conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridises to a perfectly matched probe. Typically stringent conditions will be chosen in which the salt (NaCl) concentration is about 0.02 molar at pH 7 and the temperature is at least 60° C. Lowering the salt concentration and/or increasing the temperature increases stringency.

Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“High stringency” conditions can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS. “Moderate stringency” refers to conditions equivalent to hybridization in the above described solution but at about 60-62° C. In that case the final wash is performed at the hybridization temperature in 1×SSC, 0.1% SDS. “Low stringency” refers to conditions equivalent to hybridization in the above described solution at about 50-52° C. In that case, the final wash is performed at the hybridization temperature in 2×SSC, 0.1% SDS. See also Sambrook et al. (1989) and Sambrook and Russell (2001).

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and

Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as using the Smith Waterman algorithm, are preferred. Alternatively percentage similarity or identity may be determined by searching against public databases, using algorithms such as FASTA, BLAST, etc.

Optionally, in determining the degree of “amino acid similarity”, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine.

Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

“Fungi” are herein defined as eukaryotic microorganisms and include all species of the subdivision Eumycotina (Alexopoulos, C. J., 1962, In: Introductory Mycology, John Wiley & Sons, Inc., New York). The term fungus thus includes both filamentous fungi and yeast. “Filamentous fungi” are herein defined as eukaryotic microorganisms that include all filamentous forms of the subdivision Eumycotina and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, Trichoderma, and Ustilago. “Yeasts” are herein defined as eukaryotic microorganisms and include all species of the subdivision Eumycotina that predominantly grow in unicellular form. Yeasts may either grow by budding of a unicellular thallus or may grow by fission of the organism.

The term “fungal”, when referring to a protein or nucleic acid molecule thus means a protein or nucleic acid whose amino acid or nucleotide sequence, respectively, naturally occurs in a fungus.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### DETAILED DESCRIPTION OF THE INVENTION

The commercial production of itaconic acid is reminiscent to the production of citric acid. Citric acid is commercially produced on a very large scale by Aspergillus niger, a close relative of the itaconic acid producing Aspergillus terreus. The citric acid production rate in A. niger is much more cost effective and efficient than itaconic acid production in A. terreus. The high citric acid production rate of A. niger is the result of 65 years of work examining the biochemistry, molecular biology and industrial biotechnology of citric acid production in A. niger. This has resulted is a highly efficient industrial production platform, which is highly optimized with respect to directing the metabolic flux towards citric acid. In contrast, the itaconic acid producing A. terreus is a rather underdeveloped industrial platform in comparison to A. niger.

One possible concept to improve the economic efficiency of itaconic acid production is to equip existing industrial microorganisms with the ability to convert sugars or organic acids, such as citric acid, into itaconic acid. Two metabolic pathways are suggested for the production of itaconic acid: one through decarboxylation of aconitate, an intermediate of the Krebs Cycle (Bentley and Thiessen, 1957, Biol. Chem. 223: 673-678, 689-701 and 703-720); the other pathway through condensation of acetyl-CoA and pyruvate to citramalate followed by dehydration to itaconic acid (Jakubowska and Metodiewa, 1974, Acta Microbiol. Pol., Ser. B, 6(23): 51). More recent work demonstrated that the pathway for itaconic acid production in A. terreus, paralleled that of citric acid production in A. niger with two additional steps, the dehydration of citrate to cis-aconitate and the decarboxylation of cis-aconitate to itaconic acid. The first step, the dehydration of citrate to cis-aconitate, is catalyzed by aconitate dehydratase (E.C. 4.2.1.3) and is an essential step in the Krebs Cycle. Genes encoding aconitate dehydratases are therefore present in all organisms. Since the aconitate dehydratase is already present in all organisms, expression of cis-aconitic decarboxylase, the enzyme catalysing the second step—the decarboxylation of cis-aconitate to itaconic acid—should thus be sufficient to convert selected plants or micro-organisms into an itaconic acid producers.

In a first aspect the invention relates to a polypeptide with cis-aconitic decarboxylase activity. A polypeptide with cis-aconitic decarboxylase activity (EC 4.1.1.6) is herein defined as an enzyme that catalyses the decarboxylation of cis-aconitate to itaconate and CO₂ and vice versa. Cis-aconitic decarboxylase (CAD) is also known as cis-aconitic decarboxylase, cis-aconitate carboxy-lyase or cis-aconitate carboxy-lyase (itaconate-forming). CAD enzyme activity determination is essentially performed as described by Bentley and Thiessen (1957, Biol. Chem. 223: 673-678) and Dwiarti et al. (2002, J Biosci Bioeng 94(1):29-33) and in the Examples herein. One unit (U) is one μmol of itaconic acid formed per minute under the condition the described in the Examples herein.

Polypeptides of the invention with CAD activity may be further defined by their amino acid sequence as herein described below. Likewise CADs may be defined by the nucleotide sequences encoding the enzyme as well as by nucleotide sequences hybridising to a reference nucleotide sequence encoding a CAD as herein described below.

In a second aspect the invention relates to a nucleic acid molecule comprising a nucleotide sequence encoding a polypeptide with CAD activity. A nucleotide sequence encoding a polypeptide with CAD activity preferably is selected from the group consisting of: (a) a nucleotide sequence encoding a polypeptide which comprises an amino acid sequence that has at least 40, 50, 60, 65, 70, 75, 80, 85, 90, 95, 97, 98, or 99% sequence identity with the amino acid sequence of SEQ ID NO 2 or 3; (b) a nucleotide sequence as depicted in SEQ ID NO. 1, 6 or 7; (c) a nucleotide sequence the complementary strand of which hybridises to a nucleotide sequence of (b); and, (d) a nucleotide sequence the sequence of which differs from the sequence of a nucleotide sequence of (b) or (c) due to the degeneracy of the genetic code. A nucleic acid molecule of the invention preferably is an isolated nucleic acid molecule. Examples of amino acid sequences that have at least 40% sequence identity with the amino acid sequence of SEQ ID NO 2 or 3 are given in SEQ ID NO 4 (CAD ortholog from A. oryzae) and SEQ ID NO 5 (CAD ortholog from A. niger).

A nucleic acid molecule comprising a nucleotide sequence encoding a polypeptide with CAD activity as defined above was accidentally disclosed by Kennedy et al. (1999, Science, 284:1368-1372) as pWHM1265, a plasmid comprising a part of the lovastatin biosynthesis gene cluster of A. terreus ATCC 20542. ORF15 in pWHM1265 corresponds to a nucleotide sequence encoding a polypeptide with CAD activity but was not recognised as such by Kennedy et al. (1999, supra) who indicates ORF15 to have “unknown function”. For these reasons pWHM1265 is excluded from the nucleic acid molecules of the present invention. If so required other nucleic acid molecules may be excluded from the present invention, e.g. molecules that comprise in addition to a nucleotide sequence encoding a polypeptide with CAD activity, one or more lovastatin biosynthesis genes of A. terreus or A. terreus ATCC 20542, or one or more of ORF 12, 13, 17 and 18 of A. terreus ATCC 20542 (as defined by Kennedy et al., 1999, supra) or ORFs from other A. terreus species corresponding thereto, or one or more of ORF 14 and 16 of A. terreus ATCC 20542 (as defined by Kennedy et al., 1999, supra) or ORFs from other A. terreus species corresponding thereto.

The nucleotide sequences of the invention encode polypeptides with CAD activity that may be functionally expressed in suitable host cells (see below). The nucleotide sequences of the invention preferably encode CADs that naturally occurs in certain fungi and bacteria. A preferred nucleotide sequence of the invention thus encodes a CAD with an amino acid sequence that is identical to that of a CAD that is obtainable from (or naturally occurs in) Basidiomycota or Ascomycota (formerly referred to as “Basidiomycetes” or “Ascomycetes” resp.). More preferably, the nucleotide sequence encodes a CAD that is obtainable from (or naturally occurs in) a fungus that belongs to a genus selected from Aspergillus, Gibberella (Fusarium), Pichia, Ustilago, Candida and Rhodotorula. Most preferred are nucleotide sequences encoding a CAD from Aspergillus terreus, Aspergillus itaconicus, Aspergillus oryza, Aspergillus niger, Ustilago zeae, Ustilago maydis, Rhodotorula rubra or a Candida species. Alternatively, the nucleotide sequences of the invention preferably encode CADs with an amino acid sequence that is identical to that of a CAD isomerase that is obtainable from (or naturally occurs in) a bacterium that belongs to the genera of Pseudozyma antarctica NRRL Y-7808.

It is however understood that nucleotide sequences encoding engineered forms of the fungal and bacterial CADs defined above and that comprise one or more amino acid substitutions, insertions and/or deletions as compared to the corresponding naturally occurring fungal and bacterial CADs but that are within the ranges of identity or similarity as defined herein are expressly included in the invention. Nucleotide sequences encoding CADs of the invention may e.g. be engineered in such way that the expressed protein is less susceptible to proteolytic degradation, has an improved oxygen stability or has an altered pH optimum, e.g. to a lower pH.

The nucleotide sequences of the invention, encoding polypeptides with CAD activity, are obtainable from genomic and/or cDNA of a fungus, yeast or bacterium that belongs to a phylum, class or genus as described above, using method for isolation of nucleotide sequences that are well known in the art per se (see e.g. Sambrook and Russell (2001) “Molecular Cloning: A Laboratory Manual (3^{rd} edition), Cold Spring Harbor Laboratory, Cold Spring Harbor Laboratory Press, New York). The nucleotide sequences of the invention are e.g. obtainable in a process wherein a) degenerate PCR primers are used on genomic and/or cDNA of a suitable fungus, yeast or bacterium (as indicated above) to generate a PCR fragment comprising part of the nucleotide sequences encoding the polypeptides with CAD activity; b) the PCR fragment obtained in a) is used as probe to screen a cDNA and/or genomic library of the fungus, yeast or bacterium; and c) producing a cDNA or genomic DNA comprising the nucleotide sequence encoding a polypeptide with CAD activity. Preferred fungal strains for source of cDNA or genomic DNA in a process for obtaining a nucleotide sequence of the invention are e.g. A. terreus NRRL 1960, A. terreus NIH 2624 and A. terreus ATCC 20542.

To increase the likelihood that the CAD is expressed at sufficient levels and in active form in the transformed host cells of the invention, the nucleotide sequence encoding these enzymes, are preferably adapted to optimise their codon usage to that of the host cell in question. The adaptiveness of a nucleotide sequence encoding an enzyme to the codon usage of a host cell may be expressed as codon adaptation index (CAI). The codon adaptation index is herein defined as a measurement of the relative adaptiveness of the codon usage of a gene towards the codon usage of highly expressed genes in a particular host cell or organism.

The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CM values range from 0 to 1, with higher values indicating a higher proportion of the most abundant codons (see Sharp and Li, 1987, Nucleic Acids Research 15: 1281-1295; also see: Jansen et al., 2003, Nucleic Acids Res. 31(8):2242-51). An adapted nucleotide sequence preferably has a CM of at least 0.2, 0.3, 0.4, 0.5, 0.6 or 0.7. Most preferred is the sequences as listed in SEQ ID NO 7, which has been codon optimised for expression in A. niger cells. For expression in plants the sequences listed in SEQ ID NO's: 10, 11 and 12 are more preferred, which have been codon optimised for expression, in particular for expression in potato and sugarbeet. SEQ ID NO's: 10 and 11 are most preferred for expression in plants because these sequences have been designed to have a higher GC content than SEQ ID NO: 12 to avoid deletion/truncation of the sequence during cloning. In one embodiment the invention therefore relates to codon optimised CAD coding sequence having a GC content higher than that of SEQ ID NO:12 or higher than 25, 30, 35, 40 or 45%. For changing GC content of a CAD coding sequence while maintaining a CM for a plant host cell that is higher than the wild type CAD coding sequence, preferably RSCU (Relative Synonymous Codon Usage) values present in plant genes found to have high transcript levels are used as described by Wang and Roossinck (2006, Plant Mol. Biol. 61(4): 699-710).

Further example of methods adaptation of codon usage in a coding nucleotide sequence are described in WO 2006/077258 and WO2008/000632.

Nucleotide sequence encoding CADs of the invention may also be optimised for mRNA instability, mRNA secondary structure, self homology, RNAi effects.

In a third aspect the invention pertains to a nucleic acid construct comprising a nucleotide sequence encoding a polypeptide with CAD activity as herein defined above, wherein the nucleotide sequence is operably linked to a promoter. Preferably, the promoter may be derived from a gene, which is highly expressed (defined herein as the mRNA concentration with at least 0.5% (w/w) of the total cellular mRNA). In another preferred embodiment, the promoter may be derived from a gene, which is medium expressed (defined herein as the mRNA concentration with at least 0.01% until 0.5% (w/w) of the total cellular mRNA).

In a further preferred embodiment, the promoter may be a promoter that is insensitive to catabolite (glucose) repression. More preferably, micro array data is used to select genes, and thus promoters of those genes, that have a certain transcriptional level and regulation. In this way one can optimally adapt the gene expression cassettes to the conditions under which it should function. These promoter fragments can be derived from many sources, i.e. different species, PCR amplified, synthetically and the like.

In the nucleic acid construct according to the invention the promoter preferably is a promoter that regulates transcription in a plant cell or a fungal cell. The nucleic acid construct according to the invention is thus preferably an expression vector for a plant cell or a fungal cell.

In a fourth aspect therefore, the present invention relates to a cell transformed with a nucleic acid molecule or construct comprising a nucleotide sequence encoding a polypeptide with CAD activity as herein defined above. The transformed cell (or host cell) may be any cell that produces citric acid and that comprises aconitate dehydratase (E.C. 4.2.1.3). The recipient cell for the nucleic acid molecule or construct comprising a nucleotide sequence encoding a polypeptide with CAD activity may be a bacterial, fungal or plant cell.

Preferred fungal cells for transformation with the nucleic acid molecules or constructs of the invention include fungal cells of a genus selected from Aspergillus, Penicillium, Candida and Yarrowia. More preferably, the fungal cell is of a species selected from Aspergillus niger, Aspergillus terreus, Aspergillus itaconicus, Penicillium simplicissimum, Penicillium expansuin, Penicillium digitatum, Penicillium italicum, Candida oleophila and Yarrowia lipolytica. Preferred strains are Aspergillus niger CBS120.49 and derived strains like NW185 and Candida oleophila ATCC 20177.

Preferred cells for transformation with the nucleic acid molecules or constructs of the invention are cells of an (micro)organisms (in particular filamentous fungi such as Aspergillus) that are able to produce citric acid at high yield and high rate from a suitable source of carbohydrate like e.g. glucose, fructose, sucrose, molasses, cassava, starch or corn. Measurement of citric acid is done by simple acid-base titration with NaOH keeping in mind that all acids are measured in this way.

To measure citric acid in the presence of other acids, HPLC is used (e.g. with lonPac AS-1 1 anion exchange column of Dionex, as described in their publicly available application note No. 123 of December 1998 “The determination of inorganic anions and organic acids in fermentation broths”, Dionex Corp., Sunnyvale, Calif.). When measured for instance by HPLC or titration, preferred (micro)organisms for transformation with the nucleic acid molecules or constructs of the invention are able to produce citric acid from sucrose at a level of at least 10, 20, 50, 100, or 200 g/l respectively. Modified microorganism capable of producing citric acid in even higher quantities of at least 300 g/l when produced by submerged fermentation starting from sucrose are disclosed in WO2007/063133, and these may also suitably be used as recipient cells for transformation with the nucleic acid constructs of the invention for the production of itaconic acid.

Nucleic acid constructs for expression of coding nucleotide sequences in fungi are well known in the art. In such constructs the nucleotide sequence encoding a polypeptide with CAD activity is preferably operably linked to a promoter that causes sufficient expression of the nucleotide sequences in the cell to confer to the cell the ability to convert cis-aconitate to itaconate and CO₂. Suitable promoters for expression of the nucleotide sequence as defined above include promoters that are insensitive to catabolite (glucose) repression and/or that do require induction. Promoters having these characteristics are widely available and known to the skilled person. Suitable examples of such promoters include e.g. promoters from glycolytic genes such as the phosphofructokinase, triose phosphate isomerase, glyceraldehyde-3-phosphate dehydrogenase, pyruvate kinase, phosphoglycerate kinase, glucose-6-phosphate isomerase from yeasts or filamentous fungi. Other useful promoters are ribosomal protein encoding gene promoters, alcohol dehydrogenase promoters, the enolase promoter, the cytochrome c 1 promoter, promoters from genes encoding amylo- or cellulolytic enzymes (glucoamylase, TAKA-amylase and cellobiohydrolase). Other promoters, both constitutive and inducible and enhancers or upstream activating sequences will be known to those of skill in the art. The promoters used in the nucleic acid constructs of the present invention may be modified, if desired, to affect their control characteristics. Preferably, the promoter used in the nucleic acid construct for expression of the CAD protein is homologous to the host cell in which the CAD protein is expressed.

In the nucleic acid construct of the invention for fungal expression, the 3′-end of the nucleotide acid sequence encoding the CAD preferably is operably linked to a transcription terminator sequence. Preferably the terminator sequence is operable in a host cell of choice. In any case the choice of the terminator is not critical; it may e.g. be from any fungal gene. Preferred terminators for filamentous fungal cells are obtained from the genes encoding A. oryzae TAKA amylase, the Penicillium chrysogenum pcbAB, pcbC and penDE terminators A. niger glucoamylase (glaA), A. nidulans anthranilate synthase, A. niger alpha-glucosidase, Aspergillus nidulans trpC gene and Fusarium oxysporum trypsin-like protease.

In the nucleic acid construct of the invention for fungal expression may further comprise a suitable leader sequence, a non-translated region of an mRNA that is important for translation by the cell. The leader sequence is operably linked to the 5′-terminus of the nucleic acid sequence encoding the CAD. Any leader sequence, which is functional in the cell, may be used in the present invention. Preferred leaders for filamentous fungal cells are obtained from the genes encoding Aspergillus oryzae TAKA amylase and Aspergillus nidulans triose phosphate isomerase and Aspergillus niger glaA.

Optionally, a selectable marker may be present in the nucleic acid construct. As used herein, the term “marker” refers to a gene encoding a trait or a phenotype which permits the selection of, or the screening for, a host cell containing the marker. The marker gene may be an antibiotic resistance gene whereby the appropriate antibiotic can be used to select for transformed cells from among cells that are not transformed. Examples of suitable antibiotic resistance markers include e.g. dihydrofolate reductase, hygromycin-B-phosphotransferase, 3′-O-phosphotransferase II (kanamycin, neomycin and G418 resistance). Although the use of antibiotic resistance markers may be most convenient for the transformation of polyploid host cells, preferably however, non-antibiotic resistance markers are used, such as auxotrophic markers (URA3, TRP1, LEU2) or the S. pombe TPI gene (described by Russell P R, 1985, Gene 40: 125-130). Alternatively, a screenable marker such as Green Fluorescent Protein, lacZ, luciferase, chloramphenicol acetyltransferase, or beta-glucuronidase may be incorporated into the nucleic acid constructs of the invention allowing screening for transformed cells.

A variety of selectable marker genes are available for use in the transformation of fungi.

Suitable markers include auxotrophic marker genes involved in amino acid or nucleotide metabolism, such as e.g. genes encoding ornithine-transcarbamylases (argB), orotidine-5′-decarboxylases (pyrG, URA3) or glutamine-amido-transferase indoleglycerol-phosphate-synthase phosphoribosyl-anthranilate isomerases (trpC), or involved in carbon or nitrogen metabolism, such as e.g. nitrate reductase (niaD) or facA, and antibiotic resistance markers such as genes providing resistance against phleomycin, bleomycin or neomycin (G418). Preferably, bidirectional selection markers are used for which both a positive and a negative genetic selection is possible. Examples of such bidirectional markers are the pyrG (URA3), facA and amdS genes. Due to their bidirectionality these markers can be deleted from transformed filamentous fungus while leaving the introduced recombinant DNA molecule in place, in order to obtain fungi that do not contain selectable markers, as is disclosed in EP-A-0 635 574, which is herein incorporated by reference. Of these selectable markers the use of dominant and bidirectional selectable markers such as acetamidase genes like the amdS genes of A. nidulans, A. niger and P. chrysogenum is most preferred, the amdS genes of A. niger and P. chrysogenum are disclosed in U.S. Pat. No. 6,548,285. In addition to their bidirectionality these markers provide the advantage that they are dominant selectable markers that, the use of which does not require mutant (auxotrophic) strains, but which can be used directly in wild type strains.

Optional further elements that may be present in the nucleic acid constructs of the invention include, but are not limited to, one or more leader sequences, enhancers, integration factors, and/or reporter genes, intron sequences, centromers, telomers and/or matrix attachment (MAR) sequences. The nucleic acid constructs of the invention may further comprise a sequence for autonomous replication, such as an ARS sequence.

Suitable episomal nucleic acid constructs may e.g. be based on the yeast 2μ or pKD1 (Fleer et al., 1991, Biotechnology 9: 968-975) plasmids. An autonomously maintained nucleic acid construct suitable for filamentous fungi may comprise the AMA1-sequence (see e.g. Aleksenko and Clutterbuck (1997), Fungal Genet. Biol. 21: 373-397). Alternatively the nucleic acid construct may comprise sequences for integration, preferably by homologous recombination (see e.g. WO98/46772), or gene replacement (see e.g. EP0 357 127). Such sequences may thus be sequences homologous to the target site for integration in the host cell's genome.

In order to promote targeted integration, the cloning vector is preferably linearised prior to transformation of the host cell. Linearization is preferably performed such that at least one but preferably either end of the cloning vector is flanked by sequences homologous to the target locus. The length of the homologous sequences flanking the target locus is preferably at least 30 bp, preferably at least 50 bp, preferably at least 0.1 kb, even preferably at least 0.2 kb, more preferably at least 0.5 kb, even more preferably at least 1 kb, most preferably at least 2 kb. Preferably, the efficiency of targeted integration into the genome of the host cell, i.e. integration in a predetermined target locus, is increased by augmented homologous recombination abilities of the host cell. Such phenotype of the cell preferably involves a deficient ku70 gene as described in WO2005/095624. WO2005/095624 discloses a preferred method to obtain a filamentous fungal cell comprising increased efficiency of targeted integration. Preferably, the DNA sequence in the cloning vector, which is homologous to the target locus is derived from a highly expressed locus meaning that it is derived from a gene, which is capable of high expression level in the filamentous fungal host cell. A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1). A number of preferred highly expressed fungal genes are given by way of example: the amylase, glucoamylase, alcohol dehydrogenase, xylanase, glyceraldehyde-phosphate dehydrogenase or cellobiohydrolase (cbh) genes from Aspergilli or Trichoderma. Most preferred highly expressed genes for these purposes are a glucoamylase gene, preferably an A. niger glucoamylase gene, an A. oryzae TAKA-amylase gene, an A. nidulans gpdA gene, a Trichoderma reesei cbh gene, preferably cbh1.

More than one copy of a nucleic acid sequence encoding the CAD may be inserted into the host cell to increase production of the gene product. This can be done, preferably by integrating into its genome copies of the DNA sequence, more preferably by targeting the integration of the DNA sequence at one of the highly expressed locus defined in the former paragraph.

Alternatively, this can be done by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent. To increase the copy number of the integrated nucleic acid constructs of the invention even more, the technique of gene conversion as described in WO98/46772 may be used.

The nucleic acid constructs of the invention can be provided in a manner known per se, which generally involves techniques such as restricting, linking, amplifying, and the like nucleic acids/nucleic acid sequences, for which reference is made to the standard handbooks, such as Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Transformation methods for filamentous fungi, such as Aspergilli, are well-known to the skilled person (Biotechnology of Filamentous fungi: Technology and Products. (1992) Reed Publishing (USA); Chapter 6: Transformation pages 113 to 156). The skilled person will recognize that successful transformation of fungi is not limited to the use of vectors, selection marker systems, promoters and transformation protocols specifically exemplified herein. Specific transformation protocols for A. niger are described in e.g. WO 99/32617 or WO 98/46772.

Another preferred recipient cell for transformation with the nucleic acid molecules or constructs of the invention is a plant cell. Expressly included invention are thus transgenic plants, plant cells or plant tissues or organs comprising a nucleic acid molecule or construct comprising a nucleotide sequence encoding a polypeptide with CAD activity as defined herein above.

In principle, any plant may be a suitable host for the nucleic acid constructs of the invention, such as monocotyledonous plants or dicotyledonous plants, for example sugar beet, sugar cane, maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), safflower, yam, cassava, tobacco (Nicotiana species), alfalfa (Medicago sativa), rice (Oryza species, e.g. O. sativa indica cultivar-group or japonica cultivar-group), forage grasses, pearl millet (Pennisetum spp. e.g. P. glaucum), tree species (Pinus, poplar, fir, plantain, etc), tea, coffea, oil palm, coconut, vegetable species, such as tomato (Lycopersicon ssp e.g. Lycopersicon esculentum), potato (Solanum tuberosum, other Solanum species), eggplant (Solanum melongena), peppers (Capsicum annuum, Capsicum frutescens), pea, zucchini, beans (e.g. Phaseolus species), cucumber, artichoke, asparagus, broccoli, garlic, leek, lettuce, onion, radish, turnip, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Gerbera species), herbs (mint, parsley, basil, thyme, etc.), woody trees (e.g. species of Populus, Salix, Quercus, Eucalyptus), fibre species e.g. flax (Linum usitatissimum) hemp (Cannabis sativa) and grasses, e.g. Miscanthus and switchgrass (Panicum species).

Typical host plants for use in the method according to the invention are plants which can easily be grown, which give a high yield of plant material per hectare and which can be easily harvested and processed. Typical host plants suitable for use in the method according to the invention include corn, wheat, rice, barley, sorghum, millets, sunflower, cassava, canola, soybean, oil palm, groundnut, cotton, sugar cane, chicory, bean, pea, cawpea, banana, tomato, beet, sugar beet, Jerusalem artichoke, tobacco, potato, sweet potato, coffee, cocoa and tea. In addition, said plants should preferably after transformation be able to produce large amounts of itaconic acid, give a high content of produced itaconic acid based on fresh plant material and preferably be able to deposit said itaconic acid in a concentrated manner in parts of the plant, preferably in tap roots or tubers, which can be easily harvested, stored and processed.

The construction of chimeric genes and nucleic acid constructs (vectors) for, preferably stable, introduction of a nucleotide sequence encoding a polypeptide with CAD activity into the genome of plant host cells is generally known in the art. To generate a chimeric gene the nucleic acid sequence encoding a CAD according to the invention is operably linked to a promoter sequence, suitable for expression in the host cells, using standard molecular biology techniques.

The promoter sequence may already be present in a vector so that the CAD nucleic sequence is simply inserted into the vector downstream of the promoter sequence. The vector is then used to transform the host cells and the chimeric gene is inserted in the nuclear genome or into the plastid, mitochondrial or chloroplast genome and expressed there using a suitable promoter (e.g., Mc Bride et al., 1995 Bio/Technology 13, 362; U.S. Pat. No. 5,693,507). In one embodiment a chimeric gene comprises a suitable promoter for expression in plant cells, operably linked thereto a nucleic acid sequence encoding a functional CAD protein according to the invention, optionally followed by a 3′ nontranslated nucleic acid sequence.

The CAD nucleic acid sequence, preferably the CAD chimeric gene, encoding a functional CAD protein, can be stably inserted in a conventional manner into the nuclear genome of a single plant cell, and the so-transformed plant cell can be used in a conventional manner to produce a transformed plant that has an altered phenotype due to the presence of the CAD protein in certain cells at a certain time. In this regard, a T-DNA vector, comprising a nucleic acid sequence encoding a CAD protein, in Agrobacterium tumefaciens can be used to transform the plant cell, and thereafter, a transformed plant can be regenerated from the transformed plant cell using the procedures described, for example, in EP 0 116 718, EP 0 270 822, PCT publication WO84/02913 and published European Patent application EP 0 242 246 and in Gould et al. (1991, Plant Physiol. 95, 426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718. Preferred T-DNA vectors each contain a promoter operably linked to CAD encoding nucleic acid sequence between T-DNA border sequences, or at least located to the left of the right border sequence. Border sequences are described in Gielen et al. (1984, EMBO J. 3, 835-845). Of course, other types of vectors can be used to transform the plant cell, using procedures such as direct gene transfer (as described, for example in EP 0 223 247), pollen mediated transformation (as described, for example in EP 0 270 356 and WO85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods such as those described methods for transforming certain lines of corn (e.g., U.S. Pat. No. 6,140,553; Fromm et al., 1990, Bio/Technology 8, 833-839; Gordon-Kamm et al., 1990, The Plant Cell 2, 603-618) and rice (Shimamoto et al., 1989, Nature 338, 274-276; Datta et al. 1990, Bio/Technology 8, 736-740) and the method for transforming monocots generally (PCT publication WO92/09696). The most widely used transformation method for dicot species is Agrobacterium mediated transformation. For cotton transformation see also WO 00/71733. Brassica species (e.g. cabbage species, broccoli, cauliflower, rapeseed etc.) can for example be transformed as described in U.S. Pat. No. 5,750,871 and legume species as described in U.S. Pat. No. 5,565,346. Musa species (e.g. banana) may be transformed as described in U.S. Pat. No. 5,792,935. Agrobacterium-mediated transformation of strawberry is described in Plant Science, 69, 79-94 (1990). Likewise, selection and regeneration of transformed plants from transformed cells is well known in the art. Obviously, for different species and even for different varieties or cultivars of a single species, protocols are specifically adapted for regenerating transformants at high frequency.

Besides transformation of the nuclear genome, also transformation of the plastid genome, preferably chloroplast genome, is included in the invention. One advantage of plastid genome transformation is that the risk of spread of the transgene(s) can be reduced. Plastid genome transformation can be carried out as known in the art, see e.g. Sidorov V A et al. 1999, Plant J. 19: 209-216 or Lutz K A et al. 2004, Plant J. 37(6):906-13, U.S. Pat. No. 6,541,682, U.S. Pat. No. 6,515,206, U.S. Pat. No. 6,512,162 or U.S. Pat. No. 6,492,578.

The CAD nucleic acid sequence is inserted in a plant cell genome so that the inserted coding sequence is downstream (i.e. 3′) of, and under the control of, a promoter which can direct the expression in the plant cell. This is preferably accomplished by inserting the chimeric gene in the plant cell genome, particularly in the nuclear or plastid (e.g. chloroplast) genome.

Preferred promoters include: the strong constitutive 35S promoters or (double) enhanced 35S promoters (the “35S promoters”) of the cauliflower mosaic virus (CaMV) of isolates CM 1841 (Gardner et al., 1981, Nucleic Acids Research 9, 2871-2887), CabbB-S (Franck et al., 1980, Cell 21, 285-294) and CabbB-JI (Hull and Howell, 1987, Virology 86, 482-493); the 35S promoter described by Odell et al. (1985, Nature 313, 810-812) or in U.S. Pat. No. 5,164,316, promoters from the ubiquitin family (e.g. the maize ubiquitin promoter of Christensen et al., 1992, Plant Mol. Biol. 18, 675-689, EP 0 342 926, see also Cornejo et al. 1993, Plant Mol. Biol. 23, 567-581), the gos2 promoter (de Pater et al., 1992 Plant J. 2, 834-844), the emu promoter (Last et al., 1990, Theor. Appl. Genet. 81, 581-588), Arabidopsis actin promoters such as the promoter described by An et al. (1996, Plant J. 10, 107.), rice actin promoters such as the promoter described by Zhang et al. (1991, The Plant Cell 3, 1155-1165) and the promoter described in U.S. Pat. No. 5,641,876 or the rice actin 2 promoter as described in WO070067; promoters of the Cassava vein mosaic virus (WO 97/48819, Verdaguer et al. 1998, Plant Mol. Biol. 37, 1055-1067), the pPLEX series of promoters from Subterranean Clover Stunt Virus (WO 96/06932, particularly the S7 promoter), a alcohol dehydrogenase promoter, e.g., pAdh1S (GenBank accession numbers X04049, X00581), and the TR1′ promoter and the TR2′ promoter (the “TR1′promoter” and “TR2′ promoter”, respectively) which drive the expression of the l′ and 2′ genes, respectively, of the T-DNA (Velten et al., 1984, EMBO J. 3, 2723-2730), the Figwort Mosaic Virus promoter described in U.S. Pat. No. 6,051,753 and in EP426641, histone gene promoters, such as the Ph4a748 promoter from Arabidopsis (PMB 8: 179-191), or others.

Alternatively, a promoter can be utilized which is not constitutive but rather is specific for one or more tissues or organs of the plant (tissue preferred/tissue specific, including developmentally regulated promoters), for example tap root preferred, fruit (or fruit development or ripening) preferred, leaf preferred, epidermis preferred, root preferred, flower tissue preferred, seed preferred, pod preferred, stem preferred, whereby the CAD gene is expressed only in cells of the specific tissue(s) or organ(s) and/or only during a certain developmental stage, for example during stem, leave or tap root development. For example, the CAD gene(s) can be selectively expressed in green tissue/aerial parts of a plant by placing the coding sequence under the control of a light-inducible promoter such as the promoter of the ribulose-1,5-bisphosphate carboxylase small subunit gene of the plant itself or of another plant, such as pea, as disclosed in U.S. Pat. No. 5,254,799 or Arabidopsis as disclosed in U.S. Pat. No. 5,034,322. The choice of the promoter is obviously determined by the phenotype one aims to achieve, as described above.

The production of itaconic acid is particularly advantageous in plant organs able to store large amounts of water soluble compounds, such as the tap roots of sugar beet or the stems of sugar cane, cereals or grasses, the tubers of cassava or potato, or the fruits of citrus, or the leaves of for example sugar beet, potato, grasses or tobacco. Therefore, a highly preferred promoter is a promoter which is active in organs and cell types which normally are capable of accumulating water soluble compounds. An organ-specific promoter can for example be the tuber-specific potato proteinase inhibitor II or GBSS promoter, a tap root-specific promoter such as a sucrose synthase or a fructan:fructan fructosyltransferase promoter or any other inducible or tissue-specific promoter.

To achieve expression in seeds, a seed specific promoter, as described in EP723019, EP255378 or WO9845461 can be used. For tuber specific expression (e.g. potatoes) a tuber or peel specific promoter is the most suitable such as the class II patatin promoter (Nap et al, 1992, Plant Mol. Biol. 20: 683-94) that specifies expression in the outer layer of the tuber, or a promoter with leaf and tuber peel expression such as the potato UBI7 promoter (Garbarino et al., 1995, Plant Physiol., 109: 1371-8). For root specific expression a promoter preferentially active in roots is described in WO00/29566. Another promoter for root preferential expression is the ZRP promoter (and modifications thereof) as described in U.S. Pat. No. 5,633,363.

Another alternative is to use a promoter whose expression is inducible, thus effecting induction of CAD gene expression, for example upon a change in temperature, wounding, microbial or insect attack, chemical treatment (e.g. substrate-inducible) etc. Examples of inducible promoters are wound-inducible promoters, such as the MPI promoter described by Cordera et al. (1994, The Plant Journal 6, 141), which is induced by wounding (such as caused by insect or physical wounding), or the COMPTII promoter (WO0056897) or the promoter described in U.S. Pat. No. 6,031,151. Alternatively the promoter may be inducible by a chemical, such as dexamethasone as described by Aoyama and Chua (1997, Plant Journal 11: 605-612) and in U.S. Pat. No. 6,063,985 or by tetracycline (TOPFREE or TOP 10 promoter, see Gatz, 1997, Annu Rev Plant Physiol Plant Mol. Biol. 48: 89-108 and Love et al. 2000, Plant J. 21: 579-88). Other inducible promoters are for example inducible by a change in temperature, such as the heat shock promoter described in U.S. Pat. No. 5,447,858, by anaerobic conditions (e.g. the maize ADH1S promoter), by light (U.S. Pat. No. 6,455,760), by pathogens (e.g. EP759085 or EP309862) or by senescence (SAG12 and SAG13, see U.S. Pat. No. 5,689,042). Obviously, there are a range of other promoters available.

A podwall specific promoter from Arabidopsis is the FUL promoter (also referred to as AGL8 promoter, WO9900502; WO9900503; Liljegren et al. 2004 Cell. 116(6):843-53)), the Arabidopsis IND1 promoter (Lijegren et al. 2004, supra.; WO9900502; WO9900503) or the dehiscence zone specific promoter of a Brassica polygalacturonase gene (WO9713856).

The CAD coding sequence is inserted into the plant genome so that the coding sequence is upstream (i.e. 5′) of suitable 3′ end transcription regulation signals (“3′ end”) (i.e. transcript formation and polyadenylation signals). Polyadenylation and transcript formation signals include those of the CaMV 35S gene (“3′ 35S”), the nopaline synthase gene (“3′ nos”) (Depicker et al., 1982 J. Molec. Appl. Genetics 1, 561-573), the octopine synthase gene (“3′ ocs”) (Gielen et al., 1984, EMBO J. 3, 835-845) and the T-DNA gene 7 (“3′ gene 7”) (Velten and Schell, 1985, Nucleic Acids Research 13, 6981-6998), which act as 3′-untranslated DNA sequences in transformed plant cells, and others.

Introduction of the T-DNA vector into Agrobacterium can be carried out using known methods, such as electroporation or triparental mating.

A CAD encoding nucleic acid sequence can optionally be inserted in the plant genome as a hybrid gene sequence whereby the CAD sequence is linked in-frame to a (U.S. Pat. No. 5,254,799; Vaeck et al., 1987, Nature 328, 33-37) gene encoding a selectable or scorable marker, such as for example the neo (or nptII) gene (EP 0 242 236) encoding kanamycin resistance, so that the plant expresses a fusion protein which is easily detectable.

Preferably, for selection purposes but also for weed control options, the transgenic plants of the invention are also transformed with a DNA encoding a protein conferring resistance to herbicide, such as a broad-spectrum herbicide, for example herbicides based on glufosinate ammonium as active ingredient (e.g. Liberty® or BASTA; resistance is conferred by the PAT or bar gene; see EP 0 242 236 and EP 0 242 246) or glyphosate (e.g. RoundUp®; resistance is conferred by EPSPS genes, see e.g. EP0 508 909 and EP 0 507 698). Using herbicide resistance genes (or other genes conferring a desired phenotype) as selectable marker further has the advantage that the introduction of antibiotic resistance genes can be avoided. Alternatively, other selectable marker genes may be used, such as antibiotic resistance genes.

As it is generally not accepted to retain antibiotic resistance genes in the transformed host plants, these genes can be removed again following selection of the transformants. Different technologies exist for removal of transgenes. One method to achieve removal is by flanking the chimeric gene with lox sites and, following selection, crossing the transformed plant with a CRE recombinase-expressing plant (see e.g. EP506763B1). Site specific recombination results in excision of the marker gene. Another site specific recombination systems is the FLP/FRT system described in EP686191 and U.S. Pat. No. 5,527,695. Site specific recombination systems such as CRE/LOX and FLP/FRT may also be used for gene stacking purposes. Further, one-component excision systems have been described, see e.g. WO9737012 or WO9500555).

When reference to “a transgenic plant cell” or “a recombinant plant cell” is made anywhere herein, this refers to a plant cell (or also a plant protoplast) as such in isolation or in tissue/cell culture, or to a plant cell (or protoplast) contained in a plant or in a differentiated organ or tissue, and these possibilities are specifically included herein. Hence, a reference to a plant cell in the description or claims is not meant to refer only to isolated cells in culture, but refers to any plant cell, wherever it may be located or in whatever type of plant tissue or organ it may be present. Also, parts removed from the recombinant plant, such as harvested fruit, tap roots, stems, tubers, seeds, cut flowers, pollen, etc. as well as cells derived from the recombinant cells, as well as seeds derived from traditional breeding (crossing, selfing, etc.) which retain the chimeric CAD gene are specifically included.

In a preferred embodiment the production of itaconic acid is advantageously located in cell organelles containing intermediates of the Krebs cycle, such as the mitochondria, the plastids (or plastid like organelles, such as the chloroplast or leucoplast), the cytosol or the vacuole, Accordingly, in the recombinant DNA according to the present invention, the nucleotide sequence encoding the CAD is preferably linked to a sequence encoding a transit peptide or targeting sequence which directs the mature CAD enzyme protein to a subcellular compartment, such as for example said the mitochondrion, plastid, cytosol of vacuole. For this purpose the proteins may be endowed with target peptides The terms “target peptide” refers to amino acid sequences which target a protein to intracellular organelles such as vacuoles, plastids, preferably chloroplasts, mitochondria, leucoplasts or chromoplasts, the endoplasmic reticulum, or to the extracellular space (secretion signal peptide).

A nucleic acid sequence encoding a target peptide may be fused (in frame) to the nucleic acid sequence encoding the amino terminal end (N-terminal end) of the protein or may replace part of the amino terminal end of the protein. In a further preferred embodiment, a CAD and an aconitate dehydratase are both targeted together to a (subcellular) compartment or organelle in the cell. This allows to create a metabolic sink which draws in the citric acid to be efficiently converted to itaconic acid.

In another preferred embodiment the cell transformed of the invention comprises one or more further genetic modifications that allow cheaper and/or more efficient production of itaconic acid. Such further genetic modification may include any modification that increases the flux of carbohydrates to citric acid including e.g. modifications as described in WO2007/063133.

Another preferred further genetic modification is a modification that increases the aconitate dehydratase (E.C. 4.2.1.3) activity in the cell. An increase in aconitate dehydratase activity may e.g. be achieved by increasing the copy number of endogenous copies of the aconitate dehydratase in the cell and/or introducing additional exogenous aconitate dehydratase genes. Nucleic acid constructs for (over)expression of aconitate dehydratase genes may in principle be similar or identical to the constructs described above for CAD expression except that the CAD coding sequence is replaced by a sequence coding for the aconitate dehydratase.

Yet another preferred further genetic modification may include modifications that allow the host cell to use pentoses such as xylose and/or arabinose as carbon- and energy source. For this purpose genes coding for xylose isomerases, xylulose kinases (as described e.g. in WO 03/062340 and WO 06/009434) and/or arabinose isomerases, a ribulokinases and ribulose-5-P-4-epimerases (as described in Wisselink et al., 2007, AEM Accepts, published online ahead of print on 1 Jun. 2007; Appl. Environ. Microbiol. doi:10.1128/AEM.00177-07; and in EP 1 499 708) are respectively introduced into the host cell.

Again another preferred further genetic modification may include transformation of the host cell with one or more expression constructs for (over)expression of the transporters encoded by ORF 14 and/or 16 of A. terreus ATCC 20542 (as defined by Kennedy et al., 1999, supra) or corresponding ORFs (orthologs) from other Aspergillus species or A. terreus strains.

In a fifth aspect the present invention relates to the use of a nucleic acid molecule or construct comprising a nucleotide sequence encoding a CAD as defined herein above, in the production of itaconic acid.

In sixth aspect the present invention relates to a process for producing itaconic acid, whereby the process comprises the steps of (a) fermenting a medium comprising a source of carbon and energy with a transformed cell as defined herein above, whereby the cell ferments the source of carbon and energy to itaconic acid, and optionally, (b) recovery of the itaconic acid.

A preferred fermentation process is an aerobic fermentation process. An aerobic fermentation process of the invention may be run under aerobic oxygen-limited conditions. Preferably, in an aerobic process under oxygen-limited conditions, the rate of oxygen consumption is at least 5.5, more preferably at least 6 and even more preferably at least 7 mmol/L/h.

The fermentation process may either be a submerged or a solid state fermentation process. Itaconic acid may be produced via submerged fermentation starting from a carbohydrate raw material such as for instance cassava and/or corn, which may be milled and mixed with water. A seed fermentation may be prepared in a separate fermenter. The liquefaction of the starch may be performed in the presence of an amylolytic enzyme such as for instance amylases, cellulases, lactases or maltases and additives and nutrients such as antifoam may be added before or during fermentation. For the main fermentation, the concentration of carbohydrate, e.g. starch, in the mix may be in the range of 150 to 200 g/l, preferably about 180 g/l. Alternatively, itaconic acid may be produced via surface fermentation starting from a carbohydrate raw material such as for instance a mix of beet and cane molasses or sucrose.

The fermentation process is preferably run at a temperature that is optimal for the cells of the invention. Thus, for most fungal cells, the fermentation process is performed at a temperature which is less than 42° C., preferably less than 38° C. For filamentous fungal cells, the fermentation process is preferably performed at a temperature which is lower than 35, 33, 30 or 28° C. and at a temperature which is higher than 20, 22, or 25° C.

Preferably in the fermentation processes of the invention, the cells stably maintain the nucleic acid constructs that confer to the cell the ability to produce itaconic acid.

Preferably in the process at least 10, 20, 50 or 75% of the cells retain the ability to produce itaconic acid after 50 generations of growth, preferably under industrial fermentation conditions.

In a solid state fermentation process (sometimes referred to as semi-solid state fermentation) the transformed host cells are fermenting on a solid medium that provides anchorage points for the fungus in the absence of any freely flowing substance. The amount of water in the solid medium can be any amount of water. For example, the solid medium could be almost dry, or it could be slushy. A person skilled in the art knows that the terms “solid state fermentation” and “semi-solid state fermentation” are interchangeable. A wide variety of solid state fermentation devices have previously been described (for review see, Larroche et al., “Special Transformation Processes Using Fungal Spores and Immobilized Cells”, Adv. Biochem. Eng. Biotech., (1997), Vol 55, pp. 179; Roussos et al., “Zymotis: A large Scale Solid State Fermenter”, Applied Biochemistry and Biotechnology, (1993), Vol. 42, pp. 37-52; Smits et al., “Solid-State Fermentation-A Mini Review, 1998), Agro-Food-Industry Hi-Tech, March/April, pp. 29-36). These devices fall within two categories, those categories being static systems and agitated systems. In static systems, the solid media is stationary throughout the fermentation process. Examples of static systems used for solid state fermentation include flasks, petri dishes, trays, fixed bed columns, and ovens. Agitated systems provide a means for mixing the solid media during the fermentation process. One example of an agitated system is a rotating drum (Larroche et al., supra). In a submerged fermentation process on the other hand, the transformed fungal host cells are fermenting while being submerged in a liquid medium, usually in a stirred tank fermenter as are well known in the art, although also other types of fermenters such as e.g. airlift-type fermenters may also be applied (see e.g. U.S. Pat. No. 6,746,862).

In a seventh aspect the invention relates to a process for producing itaconic acid, whereby the process comprises the steps of (a) growing a transgenic plant as herein defined above; (b) harvesting plant material comprising itaconic acid from the transgenic plant obtained in (a); and optionally, (c) recovery of the itaconic acid. In one embodiment the plant material comprising itaconic acid in (b) comprises at least 9, 12, 15, 20, 30, 50 or 100 mg itaconic acid per gram dry weight of the plant material. Preferably the plant material is a tuber, more preferably a tuber of a potato.

### DESCRIPTION OF THE FIGURES

FIG. 1: Chromatogram of the CFE of A. terreus NRRL 1960 on Source30Q. Solid line is 280 nm absorbance, dotted line is concentration of NaCl and the block diagram denotes the cis-aconitate decarboxylase (CAD) activity. Chromatographic eluens was collected in 10 mL fractions and concentrated to approximately 500 μL with Amicon Ultra-15 Centrifugal Filter Units and stored at −80° C.

FIG. 2: 12% SDS-PAGE of CAD active fractions.

FIG. 3: SDS-PAGE gel showing the CBB-stained protein pattern of 4 consecutive fractions of the anion-exchange column (#4-15 until #4-18). The CAD-activity is given in Units. Bands marked A-F were cut from the gel and processed further for peptide analysis. The most left lane of the gel contains molecular weight markers. The figures indicate the molecular weight in KDa.

FIG. 4. Sequence of protein ATEG_{—}09971. The peptides in colour were identified by LC-MSMS analysis after tryptic digestion of band A in FIG. 3.

FIG. 5: Development of Itaconic acid concentration in time for various A. niger transformants transformed with synthetic codon-optimised CAD gene (sCAD).

FIG. 6: Development of Itaconic acid concentration in time various A. niger transformants transformed with wild-type CAD cDNA (cCAD).

FIG. 7: Schematic representation of the different binary expression vectors containing the optimized CAD gene constructs: (A) pBIob 16 containing the mitochondrial targeting and the plant intron; (B) pBIob 17 also containing the mitochondrial targeting but without the intron, (C) pBIob 18 without the mitochondrial targeting (targeted to the cytosol) and without intron, (D) pBIob 19 with vacuolar targeting and also without intron. The construct name and size (in base pairs (bp)) are given in the centre of the scheme. On the vector backbone the spectinomycine resistance gene is located and labeled as Sm/SpR. The left and right border are labeled as RB and LB respectively. On the T-DNA the CAMV35S promoter is labeled as p35S, the terminator as T35S, the cassette for hygromycine resistance as Hyg. The Gateway recombination sites are labeled as attB1 and attB2. In pBIob 16 and 17 the mitochondrial targeting sequence is represented as CoxIV. In pBIob 19 the vacuolar targeting signal is represented as Ppi. The double optimized CAD encoding DNA is present in two different forms. In pBIob 16 the CAD encoding DNA sequence includes the catalase intron and is labeled as CAD (sequence nr. 0815088, SEQ ID NO: 10). In pBlob17, 18 and 19 the CAD gene without intron is present and labeled as CAD (sequence nr. 0815967 SEQ ID NO: 11). Important restriction enzyme recognition sites are labeled by the name of the corresponding restriction enzyme.

FIG. 8: HPLC analysis of leaf extract (panels A and B) and a tuber extract (panels C and D) of a transgenic potato plant harboring pBIob17 (A and C) compared to an untransformed plant extract (B and D). The position at which itaconic acid peak appears (retention time 15.6) is indicated by an arrow.

FIG. 9: Bar diagram showing the itaconic acid content (μg/gFW) of potato tubers (white, right bar of each histogram pair) and potato leaves (gray, left bar of each histogram pair) from different transgenic and control plants. The name given to the different plants starts with the name of the gene construct used for transformation, then a number for each individual line. The control plants are indicated using the construct name of the experiment they belong to, followed by a line specific label starting with a “C” and followed by a number Control plants are Biob16C01, Biob16c03, Biob17c04 and Biob17c05.

### EXAMPLES

### Example 1

### cis-Aconitate Decarboxylase (CAD) Activity Assay

The enzyme activity determination was essentially as described (Bentley et al., 1957 supra; Dwiarti et al., 2002, J Biosci Bioeng 94(1):29-33). 800 μl of 0.2 M sodium phosphate pH 6.5 was mixed with 100 μl 10 mM cis-aconitic acid and 100 μl protein solution and incubated for 20 till 60 min at 37° C. The reaction was stopped by the addition of 100 μl 12 M HCl. The amount of itaconic acid formed was determined by isocratic chromatography in 4 mM sulphuric acid on Bio-Rad Aminex HPX-87H column in a Dionex HPLC equipped with an UV detector at 215 nm. Calibration of the signal was accomplished by running a known amount of itaconic acid in a separate run. One unit (U) is one μmol of itaconic acid formed per minute. The same chromatographic assay was used to monitor the amount of itaconic acid formed in the broth of shake flasks or fermenter cultures as being indicative for cis-aconitate decarboxylase (CAD) induction. The protein concentration was measured according to Bradford with the Bio-Rad protein assay (Bradford, Anal Biochem 1976; 72:248-54).

### Example 2

### Fermentation and Induction of Itaconic Acid Production in Aspergillus Terreus NRRL 1960

Aspergillus terreus NRRL 1960 was acquired from Centraal Bureau voor Schimmelcultures, Baarn, the Netherlands. Spores were inoculated on plates of Complete Medium and grown for four days at 30° C. and fresh spores were harvested in 0.9% NaCl 0.005% Tween-80.

Pre-cultures were grown by inoculating spores (10⁶) into 100 mL pre-culture in 1 L flask containing (g/L): glucose, 25; MgSO₄.7H₂O, 4.5; NaCl, 0.4; ZnSO₄.7H₂O, 0.004; KH₂PO₄, 0.1; NH₄NO₃, 2.0; CSL (corn steep liquor), 0.5 and after two days a 10% inoculation was transferred to the CAD production medium essentially as described by Cros and Schneider (1993, U.S. Pat. No. 5,231,016) with the following changes (g/l): NH₄NO₃ (3) instead of urea, MgSO₄.7H₂O (1.5) and a final pH of 2.0.

Itaconic acid production was followed during the course of growth by HPLC analysis of the broth and correlated by the CAD activity in a cell free extract (CFE) of the corresponding mycelium. A typical result is shown in Table 1.

TABLE 1
Production of itaconic acid in a shake flask culture on 10% glucose and
detection of CAD activity in a CFE.

| | IA Produced (g/L) | CAD Activity (U/mL) |
|---|---|---|
| Day 1 | 0 | 0 |
| Day 2 | 3.9 | 0.78 |
| Day 3 | 7.3 | 0.88 |
| Day 4 | 10.3 | 0.49 |
| Day 5 | 17.6 | 0.51 |
| Day 6 | 21.9 | 0.65 |

Mycelium was harvested by filtering over a nylon filter (MW100 drd 15; Kabel Metaal, Zaandam, The Netherlands), washed with 0.2 M sodium phosphate pH 6.5, paper dried and stored at −80° C.

### Example 3

### Partial Purification of CAD from Itaconic Acid Producing A. Terreus

Approximately 1 g of frozen mycelium was transferred to a Teflon vessel and grinded with a metal ball for one minute using a dismembrator (Braun-Melsungen, Germany). Multiple batches of the powdered mycelium were resuspended in 10 ml 0.2 M sodium phosphate buffer pH 6.5 containing 1 mM DTT and 1 mM EDTA and allowed to hydrate at 0° C. for thirty minutes while mixing and centrifuged at 15000 g for 30 minutes at 4° C. to obtain the CFE.

In the purification of CAD the inherent instability of the protein was noticed. Reproduction of the purification described by Dwiarti et al (2002, supra) resulted in a completely inactive CAD preparation after the first purification step. To overcome this problem we adapted the purification method by the addition of potential stabilizers to the buffers (Table 2).

TABLE 2
Effect of the addition of stabilizing compounds on the CAD activity

| | Initial CAD Activity | CAD activity | Remaining |
|---|---|---|---|
| | (U/mL) | (U/mL) after 48 h | activity (%) |
| 20% w/v PEG | 0.84 | 0.78 | 93 |
| Ascorbic Acid* | 1.36 | 0.19 | 14 |
| Benzoate* | 1.36 | 0.43 | 32 |
| Na₂SO₃* | 1.48 | 1.30 | 88 |
| Control | 1.28 | 0.36 | 28 |
| w/o centrifugation | 1.51 | 0.20 | 13 |
| of cell suspension | | | |

*Final concentration of 20 mM.

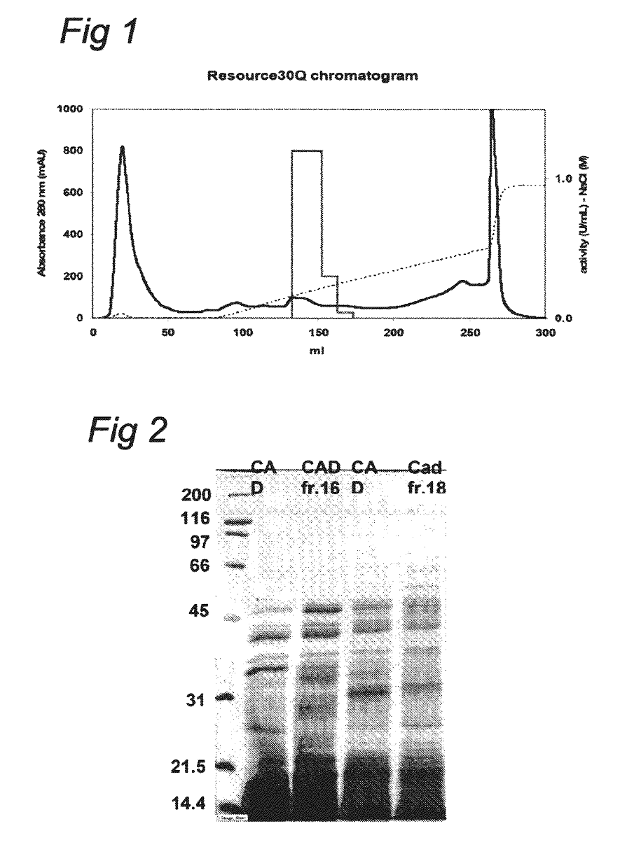

The partial purification of CAD was established by re-suspension 10 g of mycelium powder in 10 ml 50 mM Bis-TRIS, 1 mM DTT, 3 mM EDTA and 10 mM Na₂SO₃ at pH 6.9. The cleared supernatant was applied on a 19 ml Source30Q column attached to an ÄKTA explorer100 operated at 4° C. and eluted with an increasing gradient of sodium chloride (see FIG. 1).

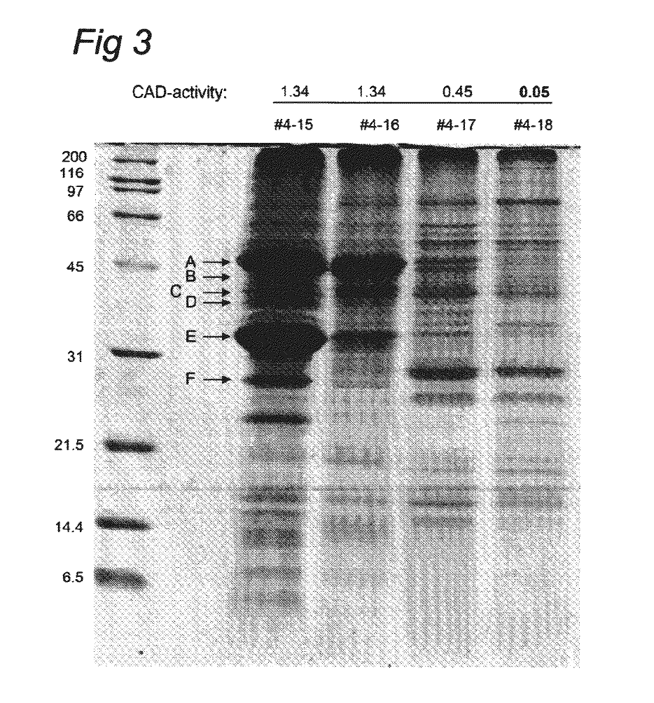

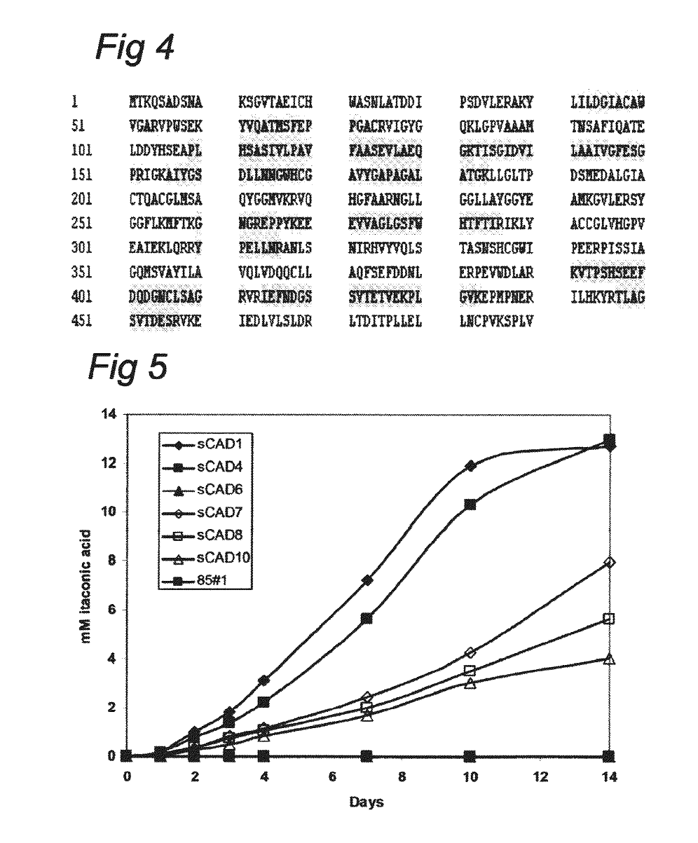

The preparation containing the partially purified CAD protein was analyzed by SDS-PAGE. For this 40 μL of the protein samples were combined with 10 μL of sample buffer (0.3 M TRIS-C1, 5% SDS, 50% glycerol and 1 mg/ml Bromphenolblue pH 8 with freshly added 100 mM DTT) at 0° C. After heating of the samples for 3 minutes at 99° C. the samples were analyzed by SDS-PAGE followed by Coomassie Brilliant Blue staining, resulted in a gel as shown in FIG. 2. The pattern of protein bands clearly shows proteolytic degradation of protein. Adapting the protocol by diluting the protein sample with sample buffer at 99° C. and immediate heating gives similar results. To solve the problem of proteolytic degradation of the protein preparation, the proteins were first precipitated with 10% TCA at 0° C. After a 5 min centrifugation (Eppendorf centrifuge) at room temperature pellets were washed with 200 μl, of ice cold acetone and dried for 5 seconds at 99° C. Protein samples were then immediately dissolved in 20 μL 5 times diluted sample buffer and heated for 3 minutes at 99° C., resulting in a gel as shown in FIG. 3 (Example 4).

### Example 4

### MS Analysis and Amino Acid Sequence of Partially Purified CAD

Protein fractions of the anion-exchange column showing CAD activity were analyzed by SDS-PAGE using a 15% (w/v) acrylamide gel. FIG. 3 shows a typical protein pattern of four consecutive fractions after staining the gel with Coomassie BB R-250. In addition to the two major bands at approx. 33 and 46 kDa (indicated by arrow A and F in FIG. 3), fraction #4-15 contained many minor bands. The intensity of the 46 kDa band correlates well with the measured CAD activity in the four fractions, being highest in fraction #4-15 and #4-16.

For mass spectrometric analysis the bands marked A-F, ranging in molecular mass between 28 and 46 kDa, were cut from the SDS-PAGE gel and sliced into 1 mm³-pieces. After destaining, the proteins were reduced with DTT and alkylated with iodoacetamide. Gel pieces were dried under vacuum, and swollen in 0.1 M NaHCO₃ containing sequence-grade porcine trypsin (10 ng/μl, Promega). After digestion at 37° C. overnight, peptides were extracted from the gel with 50% acetonitrile (ACN), 5% formic acid (FA), lyophilized, redissolved in 0.1% FA, and analyzed by LC-MS.

### Q-TOF LC-MSMS

The tryptic digests were analysed by LC-MSMS using an Ettan™ MDLC system (GE Healthcare) in high-throughput configuration directly connected to a Q-TOF-2 Mass Spectrometer (Waters Corporation, Manchester, UK). Samples (5 μl) were loaded on 5 mm×300 μm ID Zorbax™ 300 SB C18 trap columns (Agilent Technologies), and the peptides were separated on 15 cm×100 μm ID Chromolith CapRod monolithic C18 capillary columns at a flow rate of approx. 1 μl/min. Solvent A contained an aqueous 0.1% FA solution and solvent B contained 84% ACN in 0.1% FA. The gradient consisted of isocratic conditions at 5% B for 10 min, a linear gradient to 30% B over 40 min, a linear gradient to 100% B over 10 min, and then a linear gradient back to 5% B over 5 min.

MS analyses were performed in positive mode using ESI with a NanoLockSpray source. As lock mass, [Glu¹]fibrinopeptide B (1 pmol/μl) (Sigma) was delivered from a syringe pump (Harvard Apparatus, USA) to the reference sprayer of the NanoLockSpray source at a flow rate of 1 μl/min. The lock mass channel was sampled every 10 s. LC-MSMS was performed with the Q-TOF-2 operating in MS/MS mode for data dependent acquisition (DDA) of MS/MS peptide fragmentation spectra.

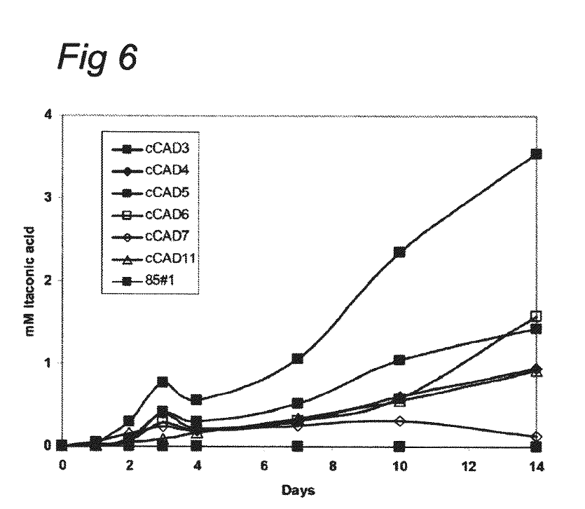

The mass spectrometer was programmed to determine charge states of the eluting peptides, and to switch from the MS to the MS/MS mode for z≧2+ at the appropriate collision energy for Argon gas-mediated CID. Each resulting MS/MS spectrum contained sequence information of a single peptide. Processing and database searching of MS/MS data sets was performed using Protein Lynx Global Server V2.3 (Waters Corporation) and the NCBI non-redundant protein database, taking fixed (carbamidomethyl) and variable (oxidation) modifications into account. The sequencing results of the protein bands marked A-F (FIG. 3) are summarized in Table 3. For each of the bands at least 3 peptide sequences were obtained that could be assigned to a protein in the Aspergillus terreus protein database. A good correlation was found between the theoretical molecular mass of the identified proteins (Table 3) and the estimated molecular mass based on the relative position of the protein in the SDS-PAGE gel (FIG. 3). Sequencing of band D revealed 5 peptide hits with thioredoxin reductase and 4 peptide hits with fructose bisphosphate aldolase, indicating that both proteins co-migrated during SDS-PAGE. As mentioned above, due to its high abundance in the two fractions containing the highest CAD-activity, band A was considered as a good candidate for representing the CAD protein. Quering the NCBI nr database with the ten peptide sequences found for band A resulted in a match to an “uncharacterized protein involved in propionate catabolism” (gi|115385453 or GeneID: 4319646). FIG. 4 shows the sequence of this protein and (in gray) the tryptic and semi-tryptic peptide sequences identified by MSMS, yielding an overall sequence coverage of 38.5%.

TABLE 3
Identified proteins of the bands marked A-F in FIG. 3.

| | | | Coverage | Mw |
|---|---|---|---|---|
| Band | Accession Description | Peptides | (%) | (Da) |
| A | ATEG_09971 Aspergillus terreus | 10 | 38.5 | 55671 |
| | predicted protein | | | |
| B | ATEG_09478 Aspergillus terreus | 4 | 10.3 | 54356 |
| | D 3 phosphoglycerate | | | |
| | dehydrogenase 2 | | | |
| C | ATEG_04676 Aspergillus terreus | 7 | 22.2 | 54207 |
| | vacuolar protease A precursor | | | |
| D | ATEG_03181 Aspergillus terreus | 5 | 18.5 | 63640 |
| | thioredoxin reductase | | | |
| E | ATEG_04703 Aspergillus terreus | 4 | 17.2 | 58141 |
| | fructose bisphosphate aldolase | | | |
| F | ATEG_05818 Aspergillus terreus | 15 | 49.2 | 94310 |
| | hypothetical protein similar to | | | |
| | STI35 protein | | | |
| | ATEG_01095 Aspergillus terreus | 3 | 8.3 | 97085 |
| | predicted protein | | | |

### Example 5

### Expression of the A. Terreus CAD Gene in A. Niger

To isolate RNA, frozen mycelium was ground using a dismembrator (Braun-Melsungen, Melsungen, Germany). After a Trizol-cholororm extraction (Invitrogen, Breda, The Netherlands) step to remove proteins, the upper phase containing total RNA was transferred to RNeasy mini columns (Qiagen, Hilden, Germany) following the manufacturer's protocol for yeast. The RNA integrity was assessed on an Experion system (Biorad laboratories, Veenendaal, The Netherlands). 1 μg of the RNA was converted to cDNA with the Omniscript kit (Qiagen). On the cDNA a proofreading PCR was performed with the forward primer: 5′-CCGGATCcatatgaccaagcaatctgcgg-3′ and the reverse primer: 5′-CCAAGCTTTAAATTATACCAGTGGCGATTTC-3′ (SEQ ID NO's: 8 and 9, respectively; restriction sites underlined) as deduced from the ATEG_{—}09971 sequence (SEQ ID NO 1).

PCR was performed using 5 units Pfu DNA polymerase and the following cycling conditions: predenaturation for 3 minutes at 97° C., followed by 30 cycles of amplification, denaturation 30 seconds 95° C., hybridisation 45 seconds at 48° C., extension 2 minutes at 72° C. and a final incubation for 10 minutes at 72° C. The CAD amplicon was visible on gel as a weak signal at approximately 1500 bp. 5 μl, of the previous PCR reaction was reamplified under identical conditions. The amplicon was ligated in pJET1 according to CloneJET™ PCR Cloning Kit (Fermentas) and transformed in electrocompetent E. coli DH5α cells (Invitrogen) and plated on LB agar plates with 100 μg/mL ampicillin. Colonies were grown in 2.5 mL LB broth with 100 μg/mL ampicillin and plasmids isolated with the GeneJet plasmid miniprep kit from Fermentas. Isolated plasmids were screened by HindIII digestion (Invitrogen). Two plasmids with the correct sized insert were sequenced and shown to be identical but having reversed inserts. Since our cDNA is derived from Aspergillus terreus NRRL 1960 and the nucleotide sequence from Aspergillus terreus strain NIH 2624 some differences in both exist.

Based on the NIH 2624 sequence a gene was synthesized by GENEART AG with the Aspergillus terreus strain NIH 2624 amino acid sequence that is codon optimized for Aspergillus niger (SEQ ID NO 7).

The cDNA gene was excised from pJET1 by the restriction endonucleases NdeI and DraI and cloned into pAL85 (an Aspergillus niger expression plasmid wherein the coding sequence to be expressed can be cloned in a multiple cloning site 3′ of the pyruvate kinase promoter and 5′ of the trpC terminator and wherein pyrA is used as selection marker) which was cut with the same enzymes. The synthetic gene was cloned into pAL85 with the restriction enzymes NdeI and NotI. Both constructs were transformed in DH5 α and plasmids isolated and characterized by PstI digestion.

Transformation of Aspergillus niger 872.11

Aspergillus niger 872.11, that is a pyrA mutant of NW185 described by Ruijter et al, (1999 Microbiology 145: 2569-2576), protoplasts were transformed according to L. H. de Graaff (1989, “The structure and expression of the pyruvatekinase gene of Aspergillus nidulans and Aspergillus niger”, PhD thesis Agricultural University Wageningen) and plated on MMS1% glucose and 0.02% arginine plates. Spores from developed colonies were harvested and again plated on MMS glucose arginine plates. From six developed colonies for each construct spores were harvested and used to inoculate PM medium (1.2 g NaNO₃, 0.5 g KH₂PO₄, 0.2 g MgSO₄.7H₂O, 0.5 g Yeast extract and 40 μL Vishniac solution pH5) containing 5% glucose and 0.02% arginine. Aspergillus niger 872.11 transformed with pAL85 was used as a reference strain. Development of itaconic acid in these PM cultures was followed by HPLC analysis.

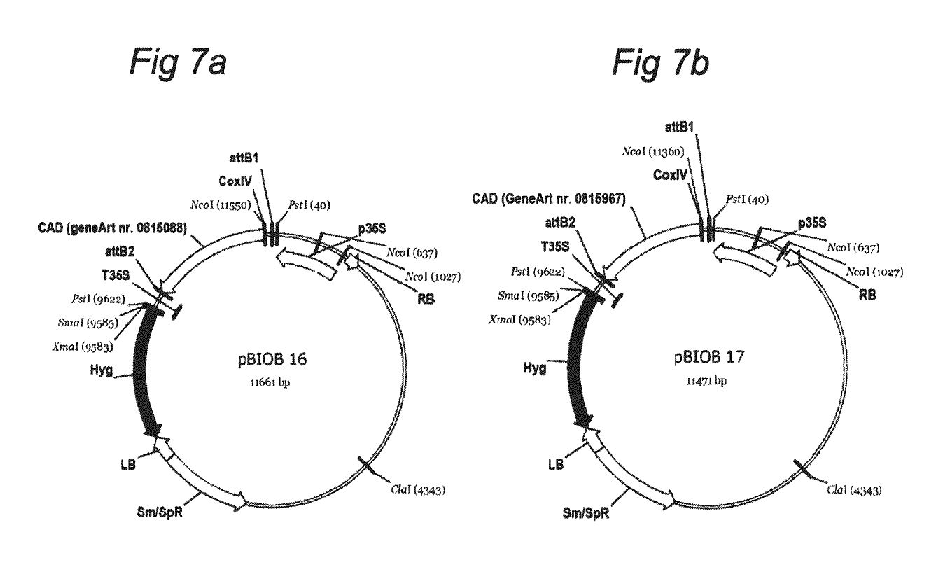

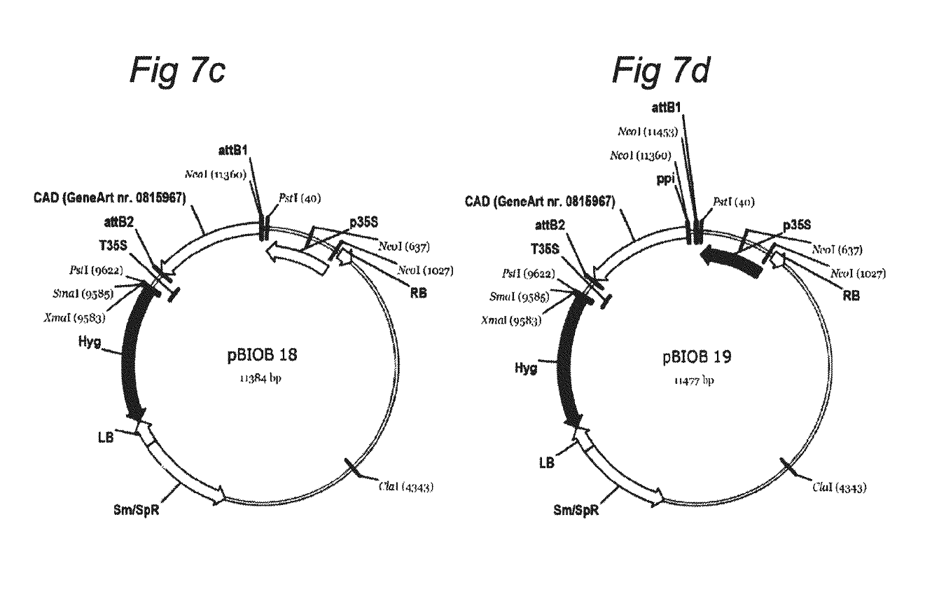

The synthetic gene (sCAD, FIG. 5) clearly gives a higher production of itaconic acid as compared to the cDNA constructs (cCAD, FIG. 6). Different transformants give rise to different production levels due to variable integration of the pAL85 constructs in to the genome of Aspergillus niger 872.11.

### Example 6

### Introduction and Expression of Aspergillus terreus CAD Genes in Plants and Accumulation of Itaconic Acid in Plants

Expression vectors were constructed to allow CAD expression in plants. For this goal, the Aspergillus terreus CAD coding sequence was optimized in two steps (optimisation of codon usage and GC content) and further also different targeting signals were fused to the CAD coding sequence to target the CAD enzyme to different plant cell compartments in order to obtain different systems for itaconic acid synthesis in plants.

### Materials and Methods

### Cloning

The CAD gene from Aspergillus terreus (WT)(CAD.pro) was cloned as described above. For expression of this microbial gene in plants, the codon usage was optimized using the codon usage tables of potato and sugarbeet, and using the proprietary GeneOptimizer® software from GeneArt. The resulting optimized DNA sequence (0804165, SEQ ID NO: 12) was synthetically produced by GeneArt (Regensburg, Germany) in two steps. Firstly, two partial CAD encoding fragments were separately cloned in pGA4 (GeneArt). The identity and sequence of the partial fragments were confirmed by DNA sequencing. In the second step, the two partial fragments were fused and ligated into pGA4 to obtain the full-length CAD encoding DNA. However, transformation of the ligation mixture into E coli resulted only in clones containing an insert with a ˜220 bp deletion at position 880 of the DNA fragment 0804165 (SEQ ID NO: 12). Repeated transformation in all cases resulted in a truncated CAD sequence. Therefore a second optimization strategy was used in addition to the first optimization strategy.

We specifically modified the region upstream of the region found to be prone to deletion, which turned out to have a 30% GC content. For changing the GC content while still optimizing the codon usage, we used RSCU (Relative Synonymous Codon Usage) values present in plant genes found to have high transcript levels (Wang and Roossinck, 2006). The resulting double optimized DNA sequence (0815967, SEQ ID NO: 11) had a higher GC content than the original sequence (0804165 SEQ ID NO: 12). The resulting optimized DNA sequence was again synthesized by GeneArt and the sequence confirmed by DNA sequencing.

Different regulatory DNA sequences were added to the CAD coding sequence to drive targeting of the expressed CAD protein to different subcellular compartments of the plant cell.

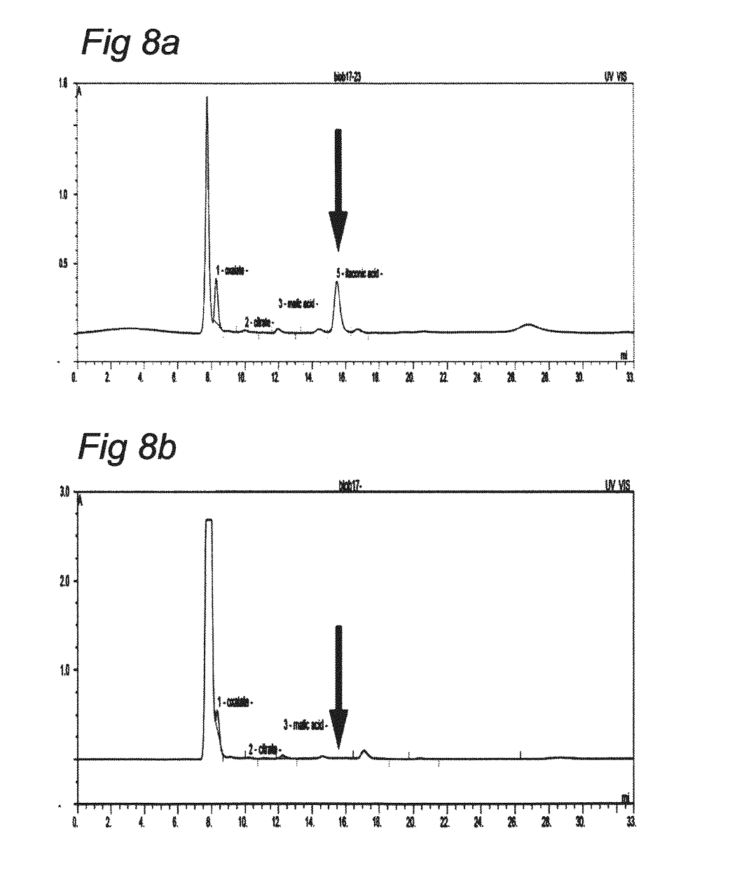

In order to target the CAD enzyme to the mitochondria, the mitochondrial targeting sequence CoxIV (Rainer H. Köhler 1997), flanked by BfuAI and NcoI restriction sites, was added upstream of the CAD coding sequence. To allow cloning into a Gateway vector system using Gateway® technology (Invitrogen®) two attb sites were included at both sides of the CoxIV-CAD fusion product (see also SEQ ID NO: 11, sequence 0815967). The full DNA sequence, comprising the mitochondrial targeting sequence CoxIV, the double optimized CAD encoding sequence, BfuAI and NcoI restriction sites and Gateway attB recombination sites, was cloned in cloning vector pMK (GeneArt) using restriction sites AscI and Pad. This full DNA sequence was eventually used for the construction of the plant transformation vector pBIob 17 (FIG. 7).

Targeting of the CAD enzyme to the cytosol of the plant cell was achieved by removing the mitochondrial targeting signal from sequence number 0815967, according to the following procedure. Two fragments were cut from the plasmid pMK0815967 (pMK vector with insert number 0815967). The first fragment containing the CAD encoding DNA sequence was cut with XhoI and NcoI. The second fragment, the backbone of the pMK vector, was cut from plasmid pMK0815967 with XhoI and BveI. Both fragments were purified and ligated to form ‘0815967-withoutCox’. This DNA sequence has eventually been used for the construction of pBIob 18 (FIG. 7).

Targeting of the CAD enzyme to the plant vacuole was achieved by ligating the vacuolar targeting fragment from the castor bean 2S albumin precursor (Ppi) (Brown, Jolliffe et al. 2003) in front of the CAD encoding DNA sequence.

As a first step, the construct pMK'0815967-withoutCox' containing the synthetic optimized CAD coding sequence with number 0815967 without the CoxIV targeting signal, was used for insertion of Ppi into the NcoI site located at the start of the CAD gene: the Ppi targeting signal had two NcoI-compatible sites at both ends. The resulting DNA fragment comprises attB recombination sites, the vacuolar targeting signal and the double optimized CAD encoding DNA. This DNA sequence was eventually used for the construction of pBIob 19 (FIG. 7).

A part of a still further optimization strategy, for example to prevent possible formation of secondary structures in the DNA and to prevent expression of the gene in E. coli or Agrobacterium tumefaciens, the CAD coding sequence was modified by inserting a plant intron into the CAD encoding DNA. Here we used the castor bean catalase intron (Suzuki, Ario et al. 1994). The catalase intron was inserted at bp1036 of the double optimized CAD coding sequence resulting in DNA sequence 0815088 (SEQ ID NO: 10). Further upstream and downstream of the catalase intron the DNA sequence of 0815088 was identical to the corresponding part of DNA sequence 0815967. After synthesis the DNA fragment 0815088 was cloned into pMK (GeneArt). This DNA sequence has eventually been used for the construction of pBIob 16 (FIG. 7).

For further cloning, the four DNA sequences 0815967, 0815967 without mitochondrial targeting signal, 0815967 with vacuolar targeting signal, and 0815088 were recombined into pDonR207 using Gateway® BP Clonase® enzyme mix (Invitrogen). The resulting entry vectors were used for transformation of E. coli Dh5a by electroporation (Maniatis et al, 1982). Subsequently, the resulting entry vectors were recombined to pH7WG2.0 (Karimi, Inzé et al. 2002) using Gateway® LR Clonase® enzyme mix (Invitrogen). This pH7WG2.0 vector contains an expression cassette driven by the cauliflower mosaic virus p35S and further contains the terminator t35S also from the Cauliflower mosaic virus 35S gene. The resulting binary vectors were called pBIob 16, pBIob 17, pBIob 18 and pBIob 19. Plasmid pBIob 16 harbours the optimised CAD gene containing an intron and with mitochondrial targeting; pBIob17 harbours the CAD gene without intron, but with mitochondrial targeting; pBIob18 harbours the CAD gene without intron and without targeting signals, which normally results in cytosolic localisation of the protein; and pBIob19 harbours the CAD gene without intron and with vacuolar targeting. In pBIob 16 and 17 the mitochondrial targeting sequence is represented as CoxIV. In pBIob 19 the vacuolar targeting signal is represented as Ppi. In pBIob16 the CAD gene sequence including the catalase intron is labeled as CAD nr. 0815088 (SEQ ID NO: 10). In pBIob17, 18 and 19 the Cad gene without intron is present and labeled as CAD nr. 0815967 (see also SEQ ID NO: 11 and FIG. 7). All constructs were used for transformation of Escherichia coli DH5α (Invitrogen, Breda, The Netherlands). The binary vectors were introduced into Agrobacterium tumefaciens strain AGL0 using transformation by high voltage electroporation (Wen-jun and Forde 1989).

SEQ ID No's: 10, 11 and 12 depict the synthetic DNA sequences 0815088, 0815967 and 0804165 containing the plant double-optimized Aspergillus terreus CAD sequence combined with restriction sites, attB recombination sites, with and without intron sequence and targeting signals necessary for cloning, expression and correct targeting in the plant cell. The first two sequences (0815088 and 0815967) have been used in the cloning in the pBIob vectors and used for plant transformation. Sequence 0815088 contains the catalase intron sequence plus the mitochondrial targeting sequence CoxIV. Sequence 0815967 also contains the mitochondrial targeting signal, but lacks the catalase intron. The last sequence (0804165) could not be used because of the low GC content and the difficulties in cloning the sequence in an expression vector.

### Transformation of Arabidopsis

To get transgenic Arabidopsis thaliana lines harbouring the T-DNAs of the constructs pBIob 16 and pBIob 17, Arabidopsis was transformed using Agrobacterium tumefaciens mediated transformation, using the flower dip method (Clough 2004). From the mature plants seeds have been harvested.

### Transformation of Potato

To get transgenic potato lines harbouring the T-DNAs of the constructs pBIob16, 17, 18 and 19, potato was transformed using Agrobacterium tumefaciens mediated transformation. In order to get a combination of constructs expressed in one plant, co-transformations were performed using combinations of Agrobacterium tumefaciens lines: pBIob17 combined with pBIob 18, pBIob 17 with pBIob 19, and pBIob 18 in combination with pBIob19. This results in expression of CAD enzymes in more than one sub-cellular compartment.

One day before potato transformation, internodal stem segments of about 5 mm long were cut from 4-6 weeks old in vitro grown potato plants.

The stem segments were collected in liquid PACM medium and transferred onto filter paper that was soaked in 2 ml of liquid PACM and put on solid PACM medium. The plates were closed with parafilm and incubated overnight at 21° C. under long day conditions (16 hours light).

For the plant transformation, freshly grown Agrobacterium tumefaciens cultures, that were grown for 16 h at 28° C., were pelleted using centrifugation at 3500 rpm for 5 minutes. The pellet was resuspended in liquid PACM (10 times more than the culture volume). The explants were transferred from the plate into the Agrobacterium suspension containing the gene construct of interest. The explants were incubated in the Agrobacterium suspension (slowly shaking) during 10 min. Then the explants were dried on filter paper and put back on the plates. For the co-cultivation the plates were closed with parafilm and incubated at 21° C. under long day conditions (16 hours light) for two days.

After the co-cultivation the explants were transferred to selection medium (ZCV), containing the appropriate antibiotic, hygromycine. The medium was refreshed every 3 weeks. The formed shoots were collected and put on solid MS30 in order to root. Control lines were made by using an empty vector Agrobacterium AGLO strain for inoculation of potato explants. These explants were not subjected to hygromycin selection during regeneration.

The following media were used in the potato transformation protocol: PACM, containing per liter 4.4 g MS medium (Murashige and Skoog, Duchefa, Haarlem, The Netherlands), 30 g sucrose, 1 mg 2.4D, 0.5 mg kinetin and 8 g microagar, pH 5.8 with KOH. Zcv, containing per liter 4.4 g MS, 20 g sucrose and 8 g microagar, pH 5.8 with KOH, with 1 mg zeatine, 200 mg cefotaxim, 50 mg vancomycin, (15 mg hygromycin). MS30 (4.41 g MS, 30 g sucrose, pH5.8, 8 g agar per liter). Antibiotic stocks were prepared as follows: 50 mg 2.4D (or 50 mg kinetin, or 50 mg zeatin) was dissolved in 1 ml KOH (1N). Heated and filled up to 50 ml with hot milliQ. Cefotaxim 200 mg/ml in milliQ, filter sterilized. Vancomycin 100 mg/ml in milliQ, filter sterilized. Kanamycin 100 mg/ml in milliQ, filter sterilized. Rifampicilin 100 mg/ml in DMSO. Hygromycin 50 mg/ml in milliQ, filter sterilized.

Rooted hygromycin resistant transgenic plants were transferred to the greenhouse and grown under normal greenhouse conditions (16 h light, 21° C.; 8 h dark, 18° C.).

### PCR Analyses of Transgenic Plans to Confirm Transgenicity

Rooted shoots were tested for transgenicity by PCR using the REDExtract-N-Amp Plant PCR Kit from Sigma according to the protocol of the manufacturer. The DNA was extracted from young leaf tissue. The primers that were used in the PCR, were designed on the hygromycin marker gene (HTPf: CTGAACTCACCGCGACGTCTG, HTPr:TCGGCGAGTACTTCTACACAG, SEQ ID NO's: 13 and 14, respectively).

### Analysis of Organic Acid Composition of Plant Material

Ten weeks after the transfer of the transformed potato plants to the greenhouse, material of young, just unfolded composed leaves were harvested and quickly frozen in liquid nitrogen. Whole tubers were collected from 8-10 week old plants, cut into pieces and frozen in liquid nitrogen. The frozen material was ground in an IKA analytical mill and was kept frozen until extraction. Organic acids were extracted from both tuber and leaf material by adding about 200 mg of ground material to one milliliter of 10 mM sulfuric acid. This was mixed using a vortex until a homogenate was obtained. The homogenate was incubated at room temperature for 30 minutes under continuous mixing. Subsequently, the extract was mixed by vortexing again. The cell debris was separated from the extract by 14000 rpm centrifugation using an Eppendorf centrifuge and by filtration over a 22 μM filter. One hundred μL of the undiluted extract was loaded on a Dionex HPLC (see also Example 1 hereinabove). In contrast to the protocol of Example 1, the run time was 33 min. per sample.

### Identification of Itaconic Acid

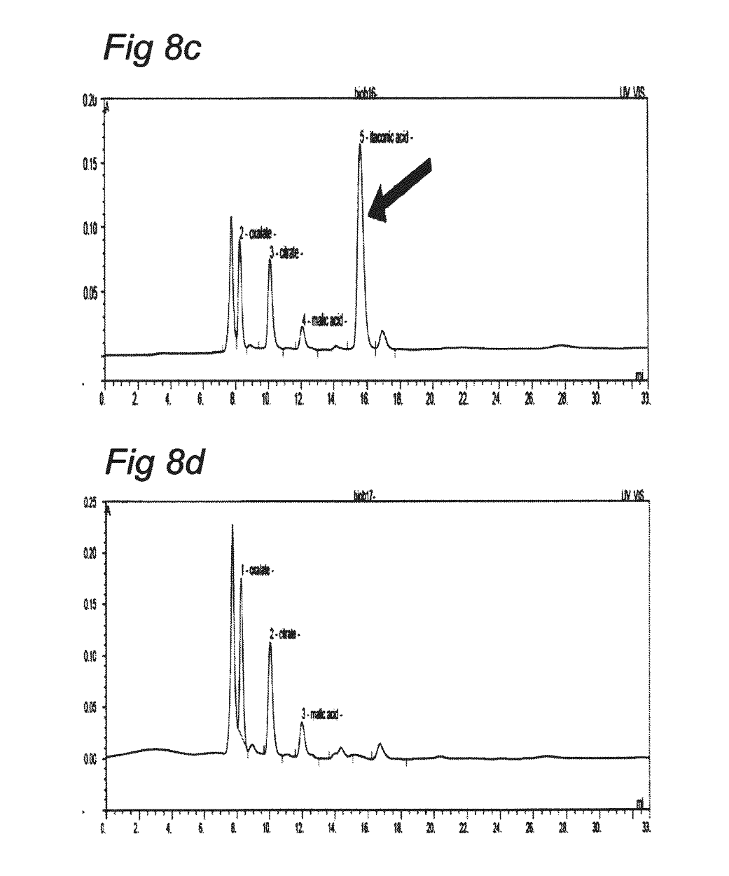

Extract from transgenic potatoes expressing the CAD encoding gene were found to contain an extra compound (peak) co-eluting with chemically pure itaconic acid obtained from Sigma (see FIG. 8). The identification of this extra peak as itaconic presence was further confirmed by spiking the transgenic potato extract with pure itaconic acid (Sigma).

LC-MS analysis was used as another identification method, according to the method described for Aspergillus niger transformed with the CAD encoding gen (this application).

### Results

All binary constructs, pBIob 16-19, described above have been used for transformation of potato. All constructs were able to induce itaconic acid synthesis in potato.

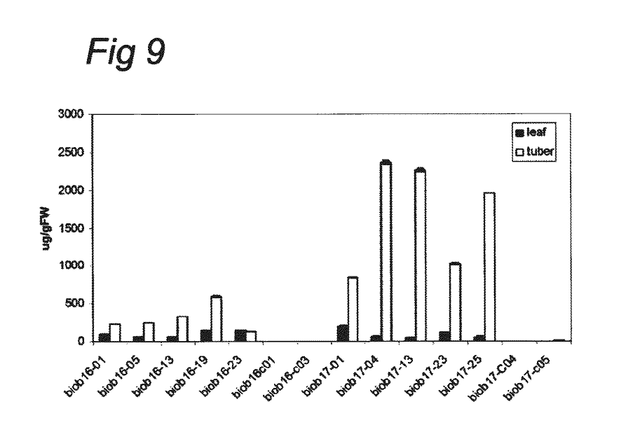

About two third of the PCR positive (transgenic) plants showed itaconic acid accumulation to various levels. FIG. 9 shows an representative example of leaves and tubers from transgenic potatoes expressing CAD. Itaconic acid was found in leaves as well as tubers of independent transformants containing different CAD constructs.

The itaconic acid level was generally higher in tubers compared to leaves, demonstrating that particularly sink organs such as tubers or taproot are suitable tissues for production and accumulation of itaconic acid (see also FIG. 9).

Plant BIOB17-04 showed the highest levels of itaconic acid in tubers, 3 mg/gFW (24 μmol/gFW). Starting from the assumption that dry weight (DW) is about 35% of potato tubers FW (fresh weight), the corresponding itaconic acid yield is at least 9 mg/gDW or at least 0.9%. None of the control plants showed any detectable amount of itaconic acid.

Young plant material of pBIob 18 transformants has been pooled and analysed. Organic acid analyses showed that the average itaconic acid concentration was 238 ug/gFW in the CAD expressing plants transformed with pBIob 18.

### REFERENCES

Brown, J. C., N. A. Jolliffe, et al. (2003). “Sequence-specific, Golgi-dependent vacuolar targeting of castor bean 2S albumin.” The Plant Journal 36: 711-719.
Clough, S. J. (2004). Floral Dip. Transgenic Plants: Methods and Protocols: 91-101.
Karimi, M., D. Inzé, et al. (2002). “GATEWAY™ vectors for Agrobacterium-mediated plant transformation.” Trends in Plant Science 7(5): 193-195.
T. Maniatis, E. P. Fritsch and J. Sambrook, Editors, (second edition ed.), Molecular Cloning: A Laboratory Manual, Cold Spring Harbor Laboratories, Cold Spring Harbor, N.Y. (1982).
Rainer H. Köhler, et al. (1997). “The green fluorescent protein as a marker to visualize plant mitochondria in vivo.” The Plant Journal 11(3): 613-621.
Suzuki, M., T. Ario, et al. (1994). “Isolation and characterization of two tightly linked catalase genes from castor bean that are differentially regulated.” Plant Molecular Biology 25(3): 507-516.
Wang, L. and M. Roossinck (2006). “Comparative analysis of expressed sequences reveals a conserved pattern of optimal codon usage in plants.” Plant Molecular Biology 61(4): 699-710.
Wen-jun, S, and B. G. Forde (1989). “Efficient transformation of Agrobacterium spp. by high voltage electroporation.” Nucl. Acids Res. 17(20): 8385.

1. A nucleic acid molecule comprising a nucleotide sequence encoding a polypeptide with cis-aconitic decarboxylase activity, wherein the nucleotide sequence is selected from the group consisting of:
(a) a nucleotide sequence encoding a polypeptide which comprises an amino acid sequence that has at least 40% sequence identity with the amino acid sequence of SEQ ID NO:2 or SEQ ID NO:3;
(b) a nucleotide sequence as depicted in of SEQ ID NO:1, SEQ ID NO:6, SEQ ID NO:7, SEQ ID NO:10, SEQ ID NO:11, or SEQ ID NO:12;
(c) a nucleotide sequence the complementary strand of which hybridizes to the nucleotide sequence of (b); and,
(d) a nucleotide sequence which differs from the sequence of (b) or (c) as a result of degeneracy of the genetic code;
with the proviso that the nucleic acid molecule is not pWHM1265.

2. A nucleic acid construct comprising a nucleotide sequence as defined in claim 1, which is operably linked to a promoter.

3. The nucleic acid construct according to claim 2, wherein the promoter is one that regulates transcription in a plant cell or a fungal cell.

4. The nucleic acid construct according to claim 2, wherein the construct is an expression vector that is expressed in a plant cell or a fungal cell.

5. A cell transformed with the nucleic acid construct according to claim 2.

6. The cell according to claim 5, which is a plant cell or a fungal cell.

7. The fungal cell according to claim 6, which is a member of a genus selected from the group consisting of Aspergillus, Penicillium, Candida and Yarrowia.

8. A transgenic plant, plant cell, plant tissue or organ comprising the nucleic acid construct according to claim 2.

9. The transgenic plant, plant cell, plant tissue or organ according to claim 8, wherein the nucleotide sequence encoding said polypeptide is operably linked to a sequence encoding a transit peptide that directs the polypeptide to a subcellular compartment selected from the group consisting of mitochondria, plastids, cytosol and vacuoles.

10. The transgenic plant, plant cell, plant tissue or organ according to claim 9, comprising a second nucleic acid construct for expression of a aconitate dehydratase polypeptide, which is operably linked to a sequence encoding a transit peptide that directs the aconitate dehydratase polypeptide to the same subcellular compartment to which the cis-aconitic decarboxylase polypeptide is directed.

11. (canceled)

12. A process for producing itaconic acid, comprising:
(a) fermenting the cells according to claim 5 in a medium comprising a carbon and an energy source in which the cell ferments the carbon and energy source to itaconic acid, and
(b) optionally, recovering the itaconic acid from the medium.

13. A process for producing itaconic acid, comprising:
(a) growing the transgenic plant according to claim 8;
(b) harvesting plant material comprising itaconic acid from the transgenic plant obtained in (a); and
(c) optionally, recovering the itaconic acid.

14. A cell transformed with the nucleic acid construct according to claim 3.

15. The fungal cell according to claim 7 which is a member of a species selected from the group consisting of Aspergillus niger, Aspergillus terreus, Aspergillus itaconicus, Penicillium simplicissimum, Penicillium expansum, Penicillium digitatum, Penicillium italicum, Candida oleophila and Yarrowia lipolytica.

16. A transgenic plant, plant cell, plant tissue or organ comprising the nucleic acid construct according to claim 3.

17. The process according to claim 12 wherein the cell is a plant cell.

18. The process according to claim 12 wherein the cell is a fungal cell.

19. The process according to claim 12 wherein the fungal cell is a member of a genus selected from the group consisting of Aspergillus, Penicillium, Candida and Yarrowia.

20. A process for producing itaconic acid, comprising:
(a) growing the transgenic plant according to claim 9;
(b) harvesting plant material comprising itaconic acid from the transgenic plant obtained in (a); and
(c) optionally, recovering the itaconic acid.

21. A process for producing itaconic acid, comprising:
(a) growing the transgenic plant according to claim 10;
(b) harvesting plant material comprising itaconic acid from the transgenic plant obtained in (a); and
(c) optionally, recovering the itaconic acid.


SEQUENCE LISTING (14 sequences)

SEQ ID NO: 1 - DNA, 1495 bp, Aspergillus terreus
  misc_feature (1)..(1494): A.terreus NRRL1960 CAD
ccggatccatatgaccaagcaatctgcggacagcaacgcaaagtcaggagttacggccgaaatatgccattgggcatccaacctggccactgacgacatcccttcggacgtattagaaagagcgaaatacctgattctcgatggtattgcatgtgcctgggttggtgcaagagtgccttggtcagagaagtatgtgcaggcaacaatgagctttgagccgccaggagcctgcagggtgattggatatgggcagaaactggggcctgttgcagcagccatgaccaattccgctttcatacaggccacagagcttgacgactaccacagcgaagcccccctacactctgcaagcatcgtcctccctgcggtctttgcagcaagtgaggtcttagccgagcaaggcaaaacaatttctggtatagatgtcattctagccgccattgtggggtttgaatctggcccgcggatcggcaaagcaatttacggatcggacctcttgaacaacggctggcattgtggagccgtgtatggtgctccagctggtgcgctggccacaggaaagctcctcggtctaactccagactccatggaagatgctctcggaatcgcgtgcacgcaagcctgtggtttaatgtcggcgcaatacggaggcatggtcaagcgcgtgcaacatggattcgcagcgcgtaatggtcttcttgggggactgttggcctatggtgggtacgaggccatgaagggtgtcctggagagatcttatggcggtttcctcaaaatgttcaccaagggcaatggcagagagcctccctacaaagaggaggaagtggtggccggtctcggttcattctggcatacctttactattcgcatcaagctctatgcctgctgcggacttgtccatggtccagtcgaagctatcgaaaagcttcagaggagataccccgagctcttgaatagagccaacctcagcaacattcgccatgtttatgtacagctttcaacagcctcgaacagtcactgtggatggataccagaggagaggcccatcagttcaatcgcagggcagatgagtgtcgcatacatcctcgccgtccagctggtcgaccagcaatgtcttctggctcagttttctgagtttgatgacaacttggagagaccagaagtgtgggatctggccaggaaggttactccatctcatagcgaagagtttgatcaagacggcaactgtctcagtgcgggtcgcgtgaggattgagttcaacgatggctcttctgttacggaaactgtcgagaagcctcttggagtcaaagagcccatgccaaacgaacggattctccacaaataccgaacccttgcgggtagcgtgacggacgaatcccgggtgaaagagattgaggatcttgtcctcagcctggacaggctcaccgacattaccccattgctggagctgcttaattgtcccgtgaaatcgccactggtataatttaaagcttgg

SEQ ID NO: 2 - protein, 490 aa, Aspergillus terreus
MTKQSADSNAKSGVTAEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAYGGYEAMKGVLERSYGGFLKMFTKGNGREPPYKEEEVVAGLGSFWHTFTIRIKLYACCGLVHGPVEAIEKLQRRYPELLNRANLSNIRHVYVQLSTASNSHCGWIPEERPISSIAGQMSVAYILAVQLVDQQCLLAQFSEFDDNLERPEVWDLARKVTPSHSEEFDQDGNCLSAGRVRIEFNDGSSVTETVEKPLGVKEPMPNERILHKYRTLAGSVTDESRVKEIEDLVLSLDRLTDITPLLELLNCPVKSPLV

SEQ ID NO: 3 - protein, 490 aa, Aspergillus terreus; identical to SEQ ID NO: 2

SEQ ID NO: 4 - protein, 482 aa, Aspergillus oryzae
MTVTDSTPEGNVTAELCNWVTELKPSDIPADVLQRAKHLLLDGIACGLVGSHVPWSEQAAKAIDDYEPEGYCSVIGYNRRYGPQAAAILNGSFIQAVELDDYHSAAPLHSASVLLPALFAAAEVQSKGHRKSVVSGLDFLVALVVGFETGPRVGSAMYGADLLSRGWHSGPVFGSPAAAAASSKLLGLSPDDTESAVGIACTQAGGLMAAQYEGMVKRVQHAFAARNGLFGALLARDGYVGIKKVFDRSYGGFLTMFTQGNGRTPQYKPEEVTTALGKEWQTTNIRVKLHACVGGCHGQIEALEKLQRNYPDRFAVDQLHNIRRITVSLSEPVFAHDGWAPEERPLTATGGQMNAAYIGAAQLVYGQVLLDQFEPHALDSDAVWSLIDKTTCVHSSEFDKPGHLCGARIVVEFNDGETVEDVVAMPKGFDPPITDDEIREKWRKLASSVIDSERLQRIENSVLSLETSADVSELLALISGEL

SEQ ID NO: 5 - protein, 541 aa, Aspergillus niger
MVTITAKSEAASATSPISSNTITTTLNGVGDPKNKEKDLQLQEKEGEEEEISKETKAYNSSNGVTSQLCTWIASLQLDDIPDSVRTRAKYLFLDGIACALVGARVPWSQKAFDAMTAFEERGKHVVIGYEERLGAIAAATLNGSWIQACEVDDYHSVAPLHSQAVVIPPLFAAAVGARDHPTTPRIIDGRTLLLASVVGFEIGPRVGMALHGTEMLAKGWHCGSVFGAPAAAGSSAKLLGLSAGQIEDAIGVAATQACGLMAAQYDGMVKRMHHGFAARNGLLGTMLAWGGYEGIKKVFERPYGGFLAMFGLGSKNTPSSKPEEVAKDLGTFWHTAEWIRLKLHACCGGIHGTIECLAEMQEMYPERLGREKLGEIKEIRIQLSDAVFHHCGWAPETRPLTPTGAQMNTAFVAASQLVDGQVLLEQFSSGKLDRDEIWELIGKTSCVHTTELDQPNIGCGALISIAFADGSQVQHSLLKPKGVDEPISNEEILEKFRRLTGGLIGVERQEKIERAVLGMEELQDVNELIELLSVNVVNPLQ

SEQ ID NO: 6 - DNA, 1529 bp, Aspergillus terreus
  misc_feature: A.terreus NIH2426 CAD
atgaccaagcaatctgcggacagcaacgcaaagtcaggagttacggccgaaatatgccattgggcatccaacctggccactgacgacatcccttcggacgtattagaaagagcgaaatacctgattctcgatggtattgcatgtgcctgggttggtgcaagagtgccttggtcagagaagtatgtgcaggcaacaatgagctttgagccgccaggagcctgcagggtgattggatatgggcaggtaagctttatccaatctagacagtctacaaagtatactgacgattctttgtatagaaactggggcctgttgcagcagccatgaccaattccgctttcatacaggccacagagcttgacgactaccacagcgaagcccccctacactctgcaagcatcgtcctccctgcggtctttgcagcaagtgaggtcttagccgagcaaggcaaaacaatttctggtatagatgtcattctagccgccattgtggggtttgaatctggcccgcggatcggcaaagcaatttacggatcggacctcttgaacaacggctggcattgtggagccgtgtatggtgctccagctggtgcgctggccacaggaaagctcctcggtctaactccagactccatggaagatgctctcggaatcgcgtgcacgcaagcctgtggtttaatgtcggcgcaatacggaggcatggtcaagcgcgtgcaacatggattcgcagcgcgtaatggtcttcttgggggactgttggcctatggtgggtacgaggccatgaagggtgtcctggagagatcttatggcggtttcctcaaaatgttcaccaagggcaatggcagagagcctccctacaaagaggaggaagtggtggccggtctcggttcattctggcatacctttactattcgcatcaagctctatgcctgctgcggacttgtccatggtccagtcgaagctatcgaaaagcttcagaggagataccccgagctcttgaatagagccaacctcagcaacattcgccatgtttatgtacagctttcaacagcctcgaacagtcactgtggatggataccagaggagaggcccatcagttcaatcgcagggcagatgagtgtcgcatacatcctcgccgtccagctggtcgaccagcaatgtcttctggctcagttttctgagtttgatgacaacttggagagaccagaagtgtgggatctggccaggaaggttactccatctcatagcgaagagtttgatcaagacggcaactgtctcagtgcgggtcgcgtgaggattgagttcaacgatggctcttctgttacggaaactgtcgagaagcctcttggagtcaaagagcccatgccaaacgaacggattctccacaaataccgaacccttgcgggtagcgtgacggacgaatcccgggtgaaagagattgaggatcttgtcctcagcctggacaggctcaccgacattaccccattgctggagctgcttaattgtcccgtgaaatcgccactggtataa

SEQ ID NO: 7 - DNA, 1551 bp, Artificial
  A.terreus NIH2624 CAD synthetic gene codon optimised for A.niger
ccggatccatatgaccaagcaatctgcggacagcaacgcaaagtcaggagttacggccgaaatatgccattgggcatccaacctggccactgacgacatcccttcggacgtattagaaagagcgaaatacctgattctcgatggtattgcatgtgcctgggttggtgcaagagtgccttggtcagagaagtatgtgcaggcaacaatgagctttgagccgccaggagcctgcagggtgattggatatgggcaggtaagctttatccaatctagacagtctacaaagtatactgacgattctttgtatagaaactggggcctgttgcagcagccatgaccaattccgctttcatacaggccacagagcttgacgactaccacagcgaagcccccctacactctgcaagcatcgtcctccctgcggtctttgcagcaagtgaggtcttagccgagcaaggcaaaacaatttctggtatagatgtcattctagccgccattgtggggtttgaatctggcccgcggatcggcaaagcaatttacggatcggacctcttgaacaacggctggcattgtggagccgtgtatggtgctccagctggtgcgctggccacaggaaagctcctcggtctaactccagactccatggaagatgctctcggaatcgcgtgcacgcaagcctgtggtttaatgtcggcgcaatacggaggcatggtcaagcgcgtgcaacatggattcgcagcgcgtaatggtcttcttgggggactgttggcctatggtgggtacgaggccatgaagggtgtcctggagagatcttatggcggtttcctcaaaatgttcaccaagggcaatggcagagagcctccctacaaagaggaggaagtggtggccggtctcggttcattctggcatacctttactattcgcatcaagctctatgcctgctgcggacttgtccatggtccagtcgaagctatcgaaaagcttcagaggagataccccgagctcttgaatagagccaacctcagcaacattcgccatgtttatgtacagctttcaacagcctcgaacagtcactgtggatggataccagaggagaggcccatcagttcaatcgcagggcagatgagtgtcgcatacatcctcgccgtccagctggtcgaccagcaatgtcttctggctcagttttctgagtttgatgacaacttggagagaccagaagtgtgggatctggccaggaaggttactccatctcatagcgaagagtttgatcaagacggcaactgtctcagtgcgggtcgcgtgaggattgagttcaacgatggctcttctgttacggaaactgtcgagaagcctcttggagtcaaagagcccatgccaaacgaacggattctccacaaataccgaacccttgcgggtagcgtgacggacgaatcccgggtgaaagagattgaggatcttgtcctcagcctggacaggctcaccgacattaccccattgctggagctgcttaattgtcccgtgaaatcgccactggtataatttaaagcttgg

SEQ ID NO: 8 - DNA, 29 bp, Artificial
  forward primer
ccggatccatatgaccaagcaatctgcgg

SEQ ID NO: 9 - DNA, 31 bp, Artificial
  backward primer
ccaagctttaaattataccagtggcgatttc

SEQ ID NO: 10 - DNA, 1894 bp, Artificial
  synthetic sequence 0815088
tgaaggaaggccgtcaaggcctaggcgcgccggtaccacaagtttgtacaaaaaagcaggctacctgccggccatgttgtcactacgtcaatctataagatttttcaagccagccacaagaactttgtgtagctctagatatctgcttcagcaaaaacccatggcaaagcaatctgctgattctaatgctaagtctggtgttactgctgagatttgccattgggcttctaatcttgctactgatgatattccttctgatgttcttgagagggctaagtaccttattcttgatggaattgcttgcgcttgggttggtgctagagttccttggagtgagaagtacgttcaagctactatgtctttcgaacctcctggtgcttgtagagttattggatacggacaaaagcttggacctgttgctgctgctatgactaattctgctttcattcaagctactgagcttgatgattaccattctgaggctcctcttcattctgcttctattgtgcttcctgctgttttcgctgcttctgaggttttggcagagcaaggaaagactatttctggaattgatgttattcttgctgctattgtgggattcgagtctggacctaggattggaaaggctatctacggatctgatcttcttaacaatggatggcattgcggagctgtttatggtgctcctgctggtgctcttgctactggaaagcttcttggacttactcctgattctatggaagatgctcttggtattgcttgcactcaagcttgcggacttatgtctgctcaatacggtggaatggttaagagggttcaacatggattcgctgctaggaatggacttcttggaggacttcttgcttatggtggatacgaggctatgaagggtgttcttgaaaggtcttacggtggattccttaagatgttcactaagggaaatggaagagagcctccttacaaagaagaggaagttgttgctggacttggatctttctggcatactttcactattaggattaagctttacgcttgctggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagcggacttgttcatggacctgttgaggctattgagaagcttcaaagaagataccctgagttgttgaacagagctaacttgtccaacatcaggcacgtgtacgtgcagctctctactgcttctaactctcactgcggatggatcccagaagagaggcctatttcttctattgctggacaaatgtctgttgcttacattcttgcagttcaacttgttgatcagcaatgccttcttgctcaattctctgagttcgatgataatcttgagaggcctgaagtttgggatcttgctaggaaggttacaccttctcattctgaagagttcgatcaagatggaaattgcctttctgctggaagagttaggattgagttcaatgatggatcttctgttactgagactgttgagaagcctcttggagttaaggaacctatgcctaatgagaggattcttcataagtacaggactcttgctggatctgttactgatgagtctagggttaaggaaattgaggatcttgttctttctcttgataggctcactgacatcactcctcttttagagcttctgaattgccctgttaagtctcctcttgtttgatgactcgagacccagctttcttgtacaaagtggtgagctcttaattaactggcctcatgggccttcctttc

SEQ ID NO: 11 - DNA, 1704 bp, Artificial
  Synthetic sequence 0815967
tgaaggaaggccgtcaaggcctaggcgcgccggtaccacaagtttgtacaaaaaagcaggctacctgccggccatgttgtcactacgtcaatctataagatttttcaagccagccacaagaactttgtgtagctctagatatctgcttcagcaaaaacccatggcaaagcaatctgctgattctaatgctaagtctggtgttactgctgagatttgccattgggcttctaatcttgctactgatgatattccttctgatgttcttgagagggctaagtaccttattcttgatggaattgcttgcgcttgggttggtgctagagttccttggagtgagaagtacgttcaagctactatgtctttcgaacctcctggtgcttgtagagttattggatacggacaaaagcttggacctgttgctgctgctatgactaattctgctttcattcaagctactgagcttgatgattaccattctgaggctcctcttcattctgcttctattgtgcttcctgctgttttcgctgcttctgaggttttggcagagcaaggaaagactatttctggaattgatgttattcttgctgctattgtgggattcgagtctggacctaggattggaaaggctatctacggatctgatcttcttaacaatggatggcattgcggagctgtttatggtgctcctgctggtgctcttgctactggaaagcttcttggacttactcctgattctatggaagatgctcttggtattgcttgcactcaagcttgcggacttatgtctgctcaatacggtggaatggttaagagggttcaacatggattcgctgctaggaatggacttcttggaggacttcttgcttatggtggatacgaggctatgaagggtgttcttgaaaggtcttacggtggattccttaagatgttcactaagggaaatggaagagagcctccttacaaagaagaggaagttgttgctggacttggatctttctggcatactttcactattaggattaagctttacgcttgctgcggacttgttcatggacctgttgaggctattgagaagcttcaaagaagataccctgagttgttgaacagagctaacttgtccaacatcaggcacgtgtacgtgcagctctctactgcttctaactctcactgcggatggatcccagaagagaggcctatttcttctattgctggacaaatgtctgttgcttacattcttgcagttcaacttgttgatcagcaatgccttcttgctcaattctctgagttcgatgataatcttgagaggcctgaagtttgggatcttgctaggaaggttacaccttctcattctgaagagttcgatcaagatggaaattgcctttctgctggaagagttaggattgagttcaatgatggatcttctgttactgagactgttgagaagcctcttggagttaaggaacctatgcctaatgagaggattcttcataagtacaggactcttgctggatctgttactgatgagtctagggttaaggaaattgaggatcttgttctttctcttgataggctcactgacatcactcctcttttagagcttctgaattgccctgttaagtctcctcttgtttgatgactcgagacccagctttcttgtacaaagtggtgagctcttaattaactggcctcatgggccttcctttc

SEQ ID NO: 12 - DNA, 1642 bp, Artificial
  Synthetic sequence 0804165
ggtaccacaagtttgtacaaaaaagcaggctacctgccggccatgttgtcactacgtcaatctataagatttttcaagccagccacaagaactttgtgtagctctagatatctgcttcagcaaaaacccatggcaaagcaatctgctgattctaatgctaagtctggtgttactgctgagatttgccattgggcttctaatcttgctactgatgatattccttctgatgttcttgagagggctaagtaccttattcttgatggaattgcttgcgcttgggttggtgctagagttccttggagtgagaagtacgttcaagctactatgtctttcgaacctcctggtgcttgtagagttattggatacggacaaaagcttggacctgttgctgctgctatgactaattctgctttcattcaagctactgagcttgatgattaccattctgaggctcctcttcattctgcttctattgtgcttcctgctgttttcgctgcttctgaggttttggcagagcaaggaaagactatttctggaattgatgttattcttgctgctattgtgggattcgagtctggacctaggattggaaaggctatctacggatctgatcttcttaacaatggatggcattgcggagctgtttatggtgctcctgctggtgctcttgctactggaaagcttcttggacttactcctgattctatggaagatgctcttggtattgcttgcactcaagcttgcggacttatgtctgctcaatacggtggaatggttaagagggttcaacatggattcgctgctaggaatggacttcttggaggacttcttgcttatggtggatacgaggctatgaagggtgttcttgaaaggtcttacggtggattccttaagatgttcactaagggaaatggaagagagcctccttacaaagaagaggaagttgttgctggacttggatctttctggcatactttcactattaggattaagctttacgcttgctgcggacttgttcatggacctgttgaggctattgagaagcttcaaagaaggtatcctgagcttcttaatagggctaatctttctaatattaggcatgtttacgttcaactttctactgcttctaattctcattgcggatggattccagaagagaggcctatttcttctattgctggacaaatgtctgttgcttacattcttgcagttcaacttgttgatcagcaatgccttcttgctcaattctctgagttcgatgataatcttgagaggcctgaagtttgggatcttgctaggaaggttacaccttctcattctgaagagttcgatcaagatggaaattgcctttctgctggaagagttaggattgagttcaatgatggatcttctgttactgagactgttgagaagcctcttggagttaaggaacctatgcctaatgagaggattcttcataagtacaggactcttgctggatctgttactgatgagtctagggttaaggaaattgaggatcttgttctttctcttgataggcttactgatattactcctcttcttgagcttttgaattgccctgttaagtctcctcttgtttgatgactcgagacccagctttcttgtacaaagtggtgagctc

SEQ ID NO: 13 - DNA, 21 bp, Artificial
  Primer HTPf
ctgaactcaccgcgacgtctg

SEQ ID NO: 14 - DNA, 21 bp, Artificial
  Primer HTPr
tcggcgagtacttctacacag